(19) United States
(12) Patent Application Publication
(10) Pub. No.: US 2020/0135321 A1
(43) Pub. Date: Apr. 30, 2020

(54) SYSTEM, METHODS, & DEVICE FOR MANAGING A PRODUCT

(71) Applicant: PILLSY, INC., Seattle, WA (US)

(72) Inventors: Jeffrey LEBRUN, Seattle, WA (US); Chukwuma ONWUNEME, Seattle, WA (US); Benjamin Otto SIPE, Seattle, WA (US)

(73) Assignee: PILLSY, INC., Seattle, WA (US)

(21) Appl. No.: 16/727,604

(22) Filed: Dec. 26, 2019

Related U.S. Application Data
(63) Continuation of application No. PCT/US2018/040150, filed on Jun. 28, 2018.
(60) Provisional application No. 62/665,504, filed on May 2, 2018.
(60) Provisional application No. 62/525,806, filed on Jun. 28, 2017.
(60) Provisional application No. 62/688,976, filed on Jun. 22, 2018.

(51) Int. Cl. G16H 20/13; G06F 21/33; A61J 7/04

(52) U.S. Cl. CPC G16H 20/13; A61J 7/04; G06F 21/33

(57) ABSTRACT

A dose management system receives a product characteristic for a product. The dose management system then associates the product with user, along with a smart monitor device that includes an embedded token. When the user thereafter installs a dose management application on a user device of the user, the dose management system receives the token, thereby authenticating the user device. The dose management system can additionally authenticate the user based on user access credentials of the user. Once the user device is authenticated, the user device, smart monitor device, and the dose management system remain in contact with each other, thereby permitting management of the product. For example, the dose management system can initiate a product refill, automatically or based on a user's request. The dose management system can also discern whether the user likely missed a dose (or not) and provide intelligent reminders to the user regarding the product.

### CROSS-REFERENCE TO RELATED APPLICATIONS

This application is a continuation of International Patent Application No. PCT/US2018/040150, filed Jun. 28, 2018, and titled “System, Methods, & Device For Managing A Product”, which claims priority to U.S. Provisional Application Ser. No. 62/688,976, filed Jun. 22, 2018, and titled “Child Resistant Closure;” to U.S. Provisional Application Ser. No. 62/665,504, filed May 2, 2018, and titled “Distributed Wireless Dose Monitoring and Reminder System;” and to U.S. Provisional Application Ser. No. 62/525,806, filed Jun. 28, 2017, and titled “Smart Medication Packaging with Pharmacy System and Method.” The entire disclosure of each of the above-identified priority applications is hereby fully incorporated herein by reference in their entirety.

### TECHNICAL FIELD

The present disclosure relates generally to a smart monitor device for managing a product, as well as to methods and systems for using the smart monitor device to manage the product. The methods and systems include authenticating a user device to work with the smart monitor device and a dose management system, managing user refills for a product, monitoring adherence to a dose schedule, and providing intelligent reminders for taking or administering the product.

### BACKGROUND

Many aspects of daily living involve the recurrent use of products that, over time, run out. And, many of these products are associated with a specific time of day or day of the week that the product should be used. For example, pool chemicals may have a dosage regimen that requires the chemicals to be added to a pool daily or weekly. And eventually, the chemicals run low or run out, triggering a re-purchase of the chemicals. It can be challenging, however, to both remember to use such products according to a dose schedule and to remember to repurchase the product before the product runs out. It can also be challenging to remember to refill products that do not follow any particular schedule but that nonetheless run out over time, such as laundry detergent.

One group of products that often requires a strict adherence to a dose schedule are medications, vitamins, and nutritional supplements. Yet a common problem is that people forget to take their medications or other pills on a regular basis, or as they are prescribed. It is estimated, for example, that average medication adherence rates are around 50% to 60% of the expected full adherence. According to the National Community Pharmacists Association, this costs about 125,000 lives and $290 billion in the United States alone.

While there are several factors behind non-adherence, numerous studies have indicated that forgetfulness is a driving factor. Further reducing patient adherence is the fact that, oftentimes, patients have numerous pill bottles and prescriptions that make keeping track of their pharmaceutical intake difficult. And while reminder systems are available to help patients remember to take their medication, for example, patients oftentimes become complicit because such reminder systems do not monitor the user's adherence and thus provide reminders for a dose the user has actually already taken.

In addition to not remembering to take a medication, people often forget to promptly manage a refill before their medication runs out, therefore causing missed doses between refills and further decreasing adherence. And while automatic refill programs can help users remember to refill their mediation, such programs are often associated with significant waste. For example, because conventional refill programs do monitor user adherence to a dosage regimen, such programs often send refills before the user actually needs the refill. This over-refilling can occur month after month, thus resulting in the user having way more medication than they need. For example, an automatic refill program may send a refill to a patient every month, regardless of whether the user actually needs the refill. The left over and unused mediation can then become a safety issue, as they may be accidentally consumed by others (e.g. children), taken beyond their expiration deadline, diverted to other users (e.g. unused narcotics), or improperly discarded. Such waste is particular an issue when a medication is to be taken on “as needed” basis, but refills are nevertheless sent before the original amount of medication is used.

In addition to forgetting medications and struggling to make refills, many people do not install and log into smartphone-based digital health applications, such as reminder applications, due to inconvenience or confusion. And, many digital health applications require more cumbersome login processes as a way to safeguard user's privacy or security. Other digital health applications require the user to input a unique code that is provided to them by their insurer or healthcare provider so that the insurer or healthcare provider can be charged by the digital health application company based on the number of people who have redeemed their unique code.

While such cumbersome onboarding processes are currently the norm in digital health applications, this inconvenience prevents many people from using these applications who may otherwise do so. And while digital health applications have grown in popularity, with over 3.7 billion downloads estimated in 2017 according to Statista.com, mobile app retention rates can be poor, with average retention reported of less than 10% after 90 days. Retention is further decreased when it becomes more difficult to securely log into a mobile application. Therefore, it is desirable and useful for users and large healthcare organizations alike to create new methods for securely and easily logging into mobile health applications that help users manage their health and that also securely sync important health information back to a healthcare organization.

Based on these and other challenges associated with maintaining and managing a product and providing user refills of the product, there is a need for a product management system that can, for example, increase adherence to a dose schedule. For example, a need exists for a dose management system that can conveniently and accurately monitor a patient's pharmaceutical consumption, and which works with a pharmacy in a way that minimizes time and effort for the patient to obtain a refill. There is also a need to provide easy refill assistance without wastefulness, as well as a need to accurately notify a user of a possible missed dose so that the user does not become complicit with inaccurate reminders. Such needs exist with medications, but also other products that require adherence to a dose schedule and refilling of the product. The present disclosure addresses these and several other needs associated with the management of a product.

### SUMMARY

In certain example aspects described herein, provided is method for managing a product. A dose management system receives one or more product characteristics for a product, the one or more product characteristics identifying the product. The dose management system also associates the product with a user and, based on the received one or more product characteristics, associates one or more management parameters with the product. A smart monitor device is also associated with the user by obtaining a first token associated with the smart monitor device and associating the first token with the user. The dose management system also authenticates a user device of the user based on the first token, thereby permitting management of the product. To authenticate the user device, for example, the dose management system receives a second token from the user device and compares the second token to a token log that includes the first token as a recorded token. By identifying a token match between the second token and the first token from the comparison of the second token to the token log, the dose management system authenticates the user device. In certain example aspects, the dose management system additionally authenticates the user device based on user credentials of the user and/or providing an authentication challenge to the user.

In certain other example aspects describe herein, provided is a method of fulfilling a refill request for a user. The dose management system, for example, associates a product that is to be refilled with a smart monitor device. The dose management system also determines one or more management parameters for the product, the one or more management parameters being determined from one or more received product characteristics of the product. The product characteristics, for example, identify a number of refills available for the product. The dose management system also associates the smart monitor device with a user by obtaining a first token associated with the smart monitor device and associating the first token with a user device of the user. The dose management system also authenticates the user device of the user based on the first token, such as by determining a token match between a second token received from the user device and a record of the first token with the dose management system. Following authentication of the user device, the dose management system receives a refill request that identifies the user. Based on the identity of the user, the dose management system determines, based on the one or more management parameters, a refill eligibility for the refill request. If the dose management system determines that the user is refill eligible, then the dose management system initiates a refill for the refill request.

In certain other example aspects described herein, provided is a method for identifying a missed dose of a product. For example, the dose management system determines one or more management parameters for a product, the one or more management parameters being determined based on one or more received product characteristics of the product. The management parameters can include, for example, a schedule of doses for the product. In addition to determining one or more management parameters, the dose management system associates a smart monitor device with a user by obtaining a first token associated with the smart monitor device and associating the first token with the user. The dose management system also authenticates a user device of the user based on the first token, such as by comparing a second token received from the user device to a token log and identifying a token match in the token log. The dose management system then verifies one or more connections between the authenticated user device and the smart monitor device and receives adherence data from the authenticated user device. The dose management system then determines, based on the received adherence data, the presence or absence of an adherence indication within a dosage time interval. An absence of the adherence indication within the dosage time interval provides an indication of a missed dose, while the presence of the adherence indication in the dosage time interval provides an indication that the dose was not missed. In certain example aspects, the user can be notified of a likely missed dose, such as via a reminder on the user device or the smart monitor device.

In certain other example aspects described herein, provided is a method of providing a refill to a user in which the user, for example, does not have to request the refill. For example, the dose management system receives an adherence indication for a user from an authenticated user device of the user. The dose management system then determines a dose count from the received adherence data. The dose count, for example, provides an estimate of the number of doses taken or administered from a container that includes or that is associated with a smart monitor device. The dose management system then compares the determined dose count to one or more management parameters. For example, the one or more management parameters are determined based on one or more received product characteristics, the one or more received product characteristics identifying the product. Based on the comparison of the determined dose count to the one or more management parameters, the dose management system then determines that a refill of the product is needed. And, in response to determining that a refill is needed, the dose management system initiates a refill for the product.

In certain other example aspects described herein, provided is a smart monitor device. The smart monitoring device includes, for example, a cap unit including a housing with a cover portion removably attached to a bottom portion, and encasing a printed circuit board and a power source in-between the cover portion and the bottom portion. The printed circuit board includes at least one sensor for detecting an open state and a closed state of the cap unit from a container unit, a wireless radio transmitter, a microprocessor, and a local memory. The smart monitor device also includes a push-plate disposed relative to the printed circuit board for movement of the push-plate to activate the sensor when the cap unit is either secured to the container unit in a closed state or is removed from the container unit in an open state. Further, the microprocessor is configured to store in memory information corresponding with the push-plate activating and/or deactivating the contact sensor, and the radio transmitter is able to wirelessly transmit the information to a user electronic computing device.

These and other aspects, objects, features and advantages of the example embodiments will become apparent to those having ordinary skill in the art upon consideration of the following detailed description of illustrated example embodiments.

### BRIEF DESCRIPTION OF THE DRAWINGS

The accompanying drawings, which are incorporated in and constitute a part of this specification, illustrate certain aspects of the instant invention and, together with the description, serve to explain, without limitation, the principles of the invention.

FIG. 1 is a block diagram depicting a system for managing a product, in accordance with certain example embodiments.

FIG. 2 is a block flow diagram depicting a method for enrolling a user and user device into a dosage management system, in accordance with certain example embodiments.

FIG. 3 is a block flow diagram depicting a method for authenticating a user device for management of a product, in accordance with certain example embodiments.

FIG. 4 is a block flow diagram depicting a method for receiving and processing a refill request, in accordance with certain example embodiments.

FIG. 5 is a block flow diagram depicting a method for monitoring user adherence to a dose schedule, in accordance with certain example embodiments.

FIG. 6 is a block flow diagram depicting a method for receiving user adherence data, in accordance with certain example embodiments.

FIG. 7 is a block flow diagram depicting a method for confirming that a dose of a product was likely missed, in accordance with certain example embodiments.

FIG. 8 is a block flow diagram depicting a method for providing a refill based on a determined dosage count, in accordance with certain example embodiments.

FIG. 9A is a top perspective view of an exemplary smart monitor device comprising a cap unit connected to a medication pill container, and able to wirelessly communicate with a user's electronic computing device.

FIG. 9B is a bottom perspective view of the inside of an exemplary cap unit of the smart monitor device.

FIG. 9C is a longitudinal cross-sectional side view of the smart monitor device of FIG. 9A taken along line C-C, illustrating the electrical-mechanical components within the cap unit with the flexible push-plate

FIG. 9D is an exploded view of the smart monitoring device of FIG. 9C.

FIG. 10A is a top plan view of an exemplary printed circuit board.

FIG. 10B is a bottom perspective view of the printed circuit board that includes a depressor switch.

FIG. 10C is an illustration of a cross-sectional view of the printed circuit board, the flexible push-plate, and the depressor switch when the cap is removed from the pill container.

FIG. 10D is an illustration of a cross-sectional view of the printed circuit board, the flexible push-plate and the depressor switch when the cap is securely fastened to the pill container or is being double tapped by the user to wirelessly, automatedly order a prescription refill.

FIGS. 11A-11G comprise electric schematic illustrations of components of the printed circuit board within the cap unit.

FIG. 11A illustrates an exemplary electrical schematic for the microprocessor comprising flash memory and antenna for short range wireless transmissions.

FIG. 11B illustrates an electric circuit schematic able to connect a button (or the internal contact sensor) to one of the general-purpose input-output pins of the SoC.

FIG. 11C is an exemplary electrical circuit schematic illustrating the LED on the printed circuit board, which can be used to notify or reminder the user.

FIG. 11D exemplary electrical circuit schematic illustrating a power circuit that is used to connect the coin cell battery.

FIG. 11E is an electrical circuit schematic of the programming port on the printed circuit board (PCB).

FIG. 11F is an electrical circuit schematic of the PCB real-time-clock (RTC).

FIG. 11G illustrates an exemplary electrical schematic for the piezo buzzer.

FIG. 12 is a block diagram, depicting a machine and a module, in accordance with certain example embodiments.

### DETAILED DESCRIPTION OF THE EXAMPLE EMBODIMENTS

### Overview

As disclosed herein, a dose management system receives a product characteristic for a product. The dose management system then associates the product with user, along with a smart monitor device that includes an embedded token. When the user thereafter installs a dose management application on a user device of the user, for example, the dose management system receives the token, thereby authenticating the user device of the user. The dose management system can additionally authenticate the user based on user access credentials of the user. Once the user device is authenticated, the user device, smart monitor device, and the dose management system can remain in contact with each other, thereby permitting management of the product. For example, the dose management system can initiate a product refill, discern whether the user likely missed a dose (or not), and provide intelligent reminders to the user regarding the product.

More particularly, in certain examples the dose management system receives one or more product characteristics for a product. Such product characteristics can identify the product, but can also provide other information regarding the product such as the name of the product, how the product is to be used or administered, and the quantity of the product. For example, a healthcare provider may send a prescription to a pharmacy for a specific medication, and the pharmacy receives the prescription for the medication. The pharmacy can then identify the product (e.g., the name of a drug) and enter the medication dose schedule into the dose management system. The pharmacist can also associate the medication with the user, such as by reading the prescription to determine the user's credentials (e.g., name, date-of-birth, username, phone number, social securing number, password, etc.) and recording the product as being connected with the user. In certain examples, the user's information can be stored in a user log of the dose management system.

In addition to associating the product with the user, in certain examples the dose management systems also determines one or more management parameters for the product that are associated with the user. For example, the dose management system reads the one or more received product characteristics to determine how to manage the product. For example, the dose management system can determine when product refills are available, the dose times for a medication, and the quantity of medication based on reading the one or more product characteristics. In certain examples, the dose management system may determine, as part of the management parameters, one or more dosage time intervals for the product, such as the time intervals in which individual doses should be taken. For example, a pharmacist can enter a start time and an end time spanning a two-hour window when a medication should be taken. In other examples, the user can enter their dosage time intervals into a user interface of a dose management application on a user device of the user.

To thereafter manage the product, the dose management system associates a smart monitor device with the user. To associate the smart monitor device with the user, each smart monitor device includes an embedded token, such as a series of numbers and/or letters that are specific to the container (i.e., a unique device identifier) that can be used to identify the specific smart monitor device. In certain examples, the smart monitor device includes audio and/visual components that can be used to communicate to the user. Before filling a container including smart monitor device with a medication, for example, a pharmacist can scan a matrix code (e.g., a QR code) or other code included with the smart monitor device—the matrix code including the token-specific information for the specific container. That is, the content of the matrix or other code matches the code for the token embedded in the hardware of the smart monitor device. For example, the matrix or other code can be printed in the same factory in which the smart monitor device is manufactured. In such examples, the pharmacist can then record the token in a token log of the dose management system as part of the user's information, thereby associating the token—and hence the smart monitor device—with the user. In other examples, the smart monitor device broadcast the embedded token to a computing device associated with the pharmacist that is configured to receive the token over a short-range connection. The pharmacist's device, for example, can then associate the token with the user.

Once a smart monitor device is associated with a user, the user installs a dose management application on a user device of the user—the application facilitating communication among the dose management system, the user device, and the smart monitor device. The user then creates a user account for the dose management system via the dose manage application, such by entering the user's credentials in a user interface of the dose management application. The dose management application can then communicate the user credentials to the dose management system. The user can also connect the user device to the smart monitor device, such as over a short-range connection, thus allowing the smart monitor device to communicate with the user device (and hence also to the dose management). For example, the smart monitor device can transmit the token to the user device and the user device can communicate the token to the dose management system via a network connection to authenticate the user device.

To authenticate the user device of the user for use with the dose management system, the dose management system uses the token and/or user credentials of the user. For example, when the dose management system receives the smart monitor device token from the user device as described herein, the dose management system reads the token and then compares the token to its stored token log. If a match is found, the user device can be authenticated and hence the user device and the smart monitor device can be enrolled with the dose management system. If a match is not found, for example, then the dose management system can notify the user of the failed authentication, such as via the dose management application of the user device.

In certain examples, for additional authentication the dose management system compares any received user access credentials with those stored in the user log of the dose management system. If a match exists, the user device can be further authenticated. That is, the dose management system can use both the token and the user credentials to authenticate the user device. If no user credential match is identified, the user can be notified of the failed authentication, such as via an alert from the installed dose management application. In certain examples, during the authentication process, an authentication challenge can be sent to the user device, such as a pin or code that the user must enter into the user device, thereby further authenticating the user device. A user response to the authentication challenge, for example, can authenticate the user.

Once the user device is authenticated and the smart device is enrolled with the dose management system, the dose management system can be used to manage numerous processes associated with taking or administering the product. For example, the dose management system can process a refill request for the user. In such examples, when a refill is needed, the user can push a button or other user control option on the smart monitor device, thereby triggering the smart monitor device to communicate a refill request to the dose management application of the user device, and hence ultimately to the dose management system via a network.

Upon receipt of the refill request, the dose management system associates the request with the user, such as by reading the request to identify the token within the request and/or reading other user access credential information of the user associated with the request. The dose management system then determines whether the user is eligible for a refill, such as by accessing and reading prior refill requests and comparing the number of prior requests to any product management parameters that identify the number of refills available. If the user is eligible for a refill request, then the dose management system can initiate the refill request, such as a medication refill, by notifying the pharmacist of the refill request. The dose management system can also update the management parameters to include the processed refill. If the user is not eligible for a refill, then in certain examples the dose management system can notify the user, such as via that dose management application, that the request has been denied. The dose management system may also relay this information to a pharmacy management system, electronic health record, or other ePrescription software, which checks for refill information and relays the refill eligibility information back to the dose management system.

In certain examples, the dose management system can additionally or alternatively manage user adherence data and, based on the adherence data, provide smart reminders to prompt the user to take or administer a dose of the product being managed. In such examples, the smart monitor device can monitor access events associated with the smart monitor device, such each open and close event of a container to which the smart monitor device is attached. For example, if the smart monitor device is included as a cap to a pill bottle, opening and/or closing of the cap can be detected, thereby providing an indication that the user was accessing the container (and presumably taking or administering a product dose from the container). The smart monitor device can then record each open and each close as access events, such as in a user access event log associated with the smart monitor device associated with the container. The smart monitor device can also time-stamp each access event, thus keeping a record of when each access event occurs.

To determine user adherence, such as user adherence to a medication, the smart monitor device can communicate the access event log to the dose management system via the dose management application of the user device. By reading and analyzing the access event log, the dose management system can determine the presence or absence of an adherence indication (and hence either adherence to a schedule for the product or a possible missed dose, respectively). For example, the dose management system can read the received access event log and compare the log to the product management parameters to determine an adherence indication, such as an access event at or near the time of a scheduled dose.

If the dose management system does not identify an adherence indication for a scheduled dose, the dose management system can, in certain examples, also verify whether the possible missed dose was more likely a missed dose or a system communication error. For example, based on the lack of an expected adherence indication, the dose management system can request connection data and adherence data from the user device associated with the smart monitor device. If no response is received from the request, the dose management system can attempt to notify the user of the connection error so that the error can be addressed. But if the dose management system receives a response, the dose management system can confirm that the network connection between the dose management system and the user device is intact, and that the missing adherence indication was not the result of a connection error between the dose management system and the user device.

In certain examples, by reading and analyzing the received connection data, the dose management system can also determine whether a connection error exists between the user device and the smart monitor device. For example, the dose management system can determine from the connection data whether the smart monitor device has been in contact with the user device. If not, the dose management system can notify the user of the error. But if the dose management system determines that the connection between the user device and the smart monitor device is (and has been) intact, the dose management system can determine that the missing adherence indication was likely not due to a connection error (and more likely a possible missed dose).

Similarly, by reading the connection data, in certain examples the dose management system can determine if a connection between the smart monitor device and the user device existed during a specific dosage time interval. For example, if a medication dose was supposed to be taken at 9:00 a.m., and the dosage time interval for that dose was 8:00 a.m. to 10:00 a.m., then the dose management system can read the connection data to ensure that the smart monitor device was connected throughout—or at some point during—the dosage time interval. If not, the dose management system can notify the user of the connection issue. But if a connection did exist during the window, the dose management system can determine that a missed dose was more likely.

In certain examples, the dose management system can also read the received adherence data to determine the time proximity of an adherence indication to a scheduled dose, thereby permitting a further determination of whether a dose was actually missed. For example, if a medication dose was scheduled at 9:00 a.m., and the dose management system receives a missing adherence indication associated with the missed dose, the dose management system can determine the connection statuses described above to ensure there was no connection issue. Once the connections are validated, however, the dose management system can then read and analyze the received adherence data to identify any adherence indication during a defined dosage window. For example, for a 9:00 a.m. scheduled dose, the dose management system may read the adherence data and identify an adherence indication at 10:07 a.m., based on the timestamps associated with the adherence indications. Hence, the dose management system can determine that a dose likely was not actually missed based on the 10:07 a.m. adherence indication. But if no adherence indications are identified within the dosage time interval, the dose management system can determine that a dose was very likely missed and thereafter notify the user and/or the user's healthcare provider of the likely missed dose.

In other examples, the dose management system can also read the adherence data to determine when and if a refill might be needed, such as without the user having to make a refill request. For example, based on the adherence indications identified in the adherence data, the dose management system can determine how many doses a user has taken or administered, such as the number of doses over the course of a month. The dose management system then compares the determined dose count to the management parameters, such as the number of scheduled doses for the product. For example, the management parameters may provide that that 30 doses have been initially provided to the user, and that each dose is to be taken daily for the next 30 days. On day 25, for example, the dose management system can determine that 25 doses have likely been taken and that hence a refill should be initiated (as the doses will soon run out).

By using and relying on the methods and system described herein, a user can easily and effectively manage a product. When used for managing a medication, for example, a user can easily and securely enroll in the dose management system. Once enrolled, the user can easily request refills via a click-button option, for example, and have their refill count monitored. And importantly, the refills can be sent when they are actually needed, thereby eliminating waste. And in certain examples, with the methods and systems provided herein, a refill can be provided automatically to the user based on indications that the user is actually taking the medication. As such, medication is not sent to the user when the user is not taking the medication, thereby further decreasing waste. Such automatic refills also increase user adherence to a dose schedule, as the user is not forgetting to request a refill.

Further, the methods and systems described herein allow the user to receive smart or intelligent reminders for a likely missed dose, thereby greatly reducing inaccurate reminders that cause the user to become complicit and ignore the reminders generally. Such capabilities greatly improve the user's adherence, thereby also benefiting the user's health and wellbeing (in the case of medications, for example). And because the methods and systems described herein can be separately used for different pill bottles, for example, the methods and systems described herein can greatly benefit the user having to manage multiple prescriptions at a time. For example, different reminders can be sent for different medications. Also, not only do the methods and systems described herein benefit the user, they also benefit the user's healthcare provider. For example, by relying on the dose management system to read, analyze, and manage adherence data for a user, the user's healthcare provider can beneficially monitor the user's adherence as a patient compliance indicator. The healthcare provider can also, via the methods and system described herein, notify the user of a missed dose.

As those skilled in the art will appreciate based on this disclosure, while the methods and systems described herein can be described in the context of a prescription or non-prescription medication, such methods and systems can be used for any product that requires—or for which it may be helpful to require—adherence to a dose schedule. For example, a user may use the methods and systems described herein to manage the administration of water treatment schedule, such as the administration of water treatment chemicals to a pool. The user can also use the methods and systems described herein to manage refills of such chemicals, without producing waste. In such examples, the user's employer or manager may also rely on the methods and systems described herein to monitor activities associated with the product. For example, the employer or manager may use and rely on the methods described herein to monitor whether a remote worker has, in fact, added chemicals to the pool. The employer or manager can also use the methods and systems described herein to determine—very accurately—when the user accessed the pool chemicals and hence likely added the chemicals to the pool. The same methods and systems can also be used to monitor whether chemicals have been dispensed for other industrial processes, such as a water treatment system. These and other benefits will become apparent to those skilled in the art based on this disclosure.

### Example System Architecture

Turning now to the drawings, in which like numerals indicate like (but not necessarily identical) elements throughout the figures, example embodiments are described in detail.

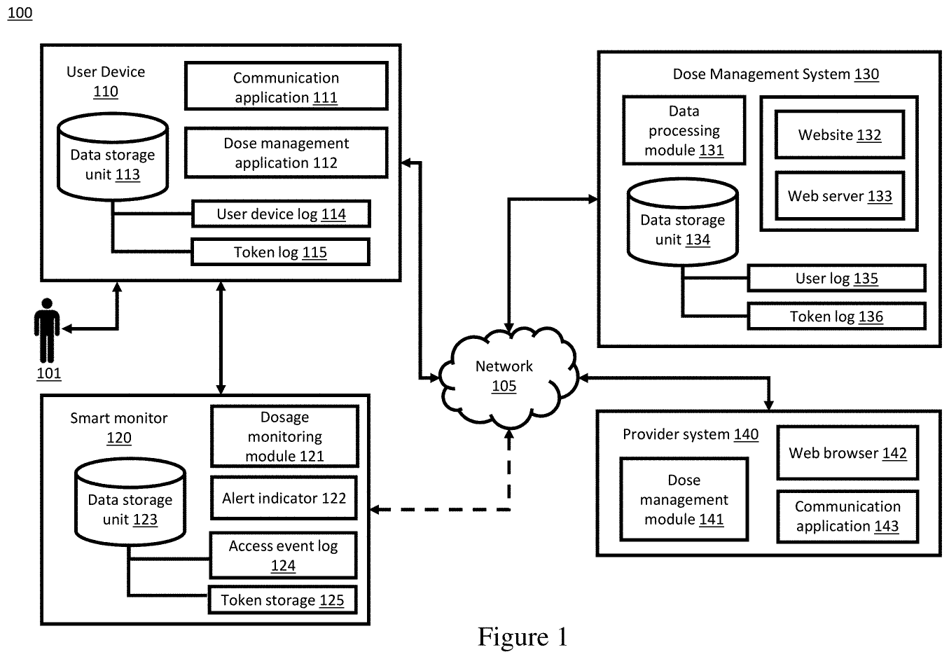

FIG. 1 is a block diagram depicting a system for managing a product, in accordance with certain example embodiments.

As described in FIG. 1, the exemplary operating system environment 100 includes a network computing user device 110, a smart monitor device 120, a dose management system 130 (or “DMS”), and, in certain examples a provider system 140. In another example embodiment, two or more of these components (including components 110, 120, 130, and 140) or parts thereof can be integrated into the same system and/or device. In certain examples, a user 101 associated with a user device 110 must make a feature selection and/or install an application on the user device 110 to obtain the benefits of the methods described herein.

Each network component 110, 120, 130, and 140 of the example operating system environment 100 includes a device having a communication module (not shown) capable of transmitting and receiving data over the network 105. Further, each component 110, 120, 130, and 140 can include a server, desktop computer, laptop computer, tablet computer, a television with one or more processors embedded therein and/or coupled thereto, smart phone, handheld computer, personal digital assistant (“PDA”), smart watch, or any other wired or wireless, processor-driven device. In the example embodiment depicted in FIG. 1, the network components 110 and 120 can be operated, for example, by end-users or consumers or other end user operators. Likewise, the network components 130 and 140 can be operated by dose management system operators and provider system operators, respectively, or other end user operators.

The network 105 includes a wired or wireless telecommunication system or device by which network components (including components 110, 130, and 140) can exchange data. For example, the network 105 can include a local area network (“LAN”), a wide area network (“WAN”), an intranet, an Internet, storage area network (“SAN”), personal area network (“PAN”), a metropolitan area network (MAN), a wireless local area network (“WLAN”), a virtual private network (“VPN”), a cellular 2G, 3G, 4G, 5G, LTE, Narrowband IoT (“NB-IoT”) or other mobile communication network, Bluetooth, a wireless short-range 2.4 GHz connection, near field communication (“NFC”), or any combination thereof or any other appropriate architecture or system that facilitates the communication of signals, data, and/or messages.

As shown in FIG. 1, the user device 110 includes a communication application 111 and associated web browser (not shown) that can interact with web servers or other computing devices and systems connected to the network 105. For example, the communication application 111 can interact with web servers or other computing devices connected to the network 105, including web servers (not shown) of the dose management system 130 and the provider system 140. In certain examples, the communication application 111 can communicate directly, as shown, with the smart monitor device 120 via short-range data transmissions, such as via Bluetooth, near field communication, decodable audio signal, wireless short-range 2.4 GHz connection, or other short-range appropriate architecture or system that facilitates the communication of signals, data, and/or messages. A user 101 can use the communication application 111 of the user device 110, for example, to view, download, upload, or otherwise access information, web pages, or other data via a distributed network 105. For example, the user 101 may use the communication application 111 to receive data and information from the smart monitor device 120 (via short-range connections) and transmit those data to the dose management system 130 and/or the provider system 140.

As shown, the user device 110 can also include a dose management application 112 that is configured to interact and communicate with the smart monitor device 120, the dose management system 130, and the provider system 140 via communication application 111. For example, the dose management application 112 can be used and configured to receive and send device connection data to dose management system 130, such as data and information related to the connections status of the user device 110 to the smart monitor device 120 (e.g., when and for how long the user device 110 and the smart monitor device 120 were or have been connected). The dose management application 112 can also be used and configured to receive and send adherence data to the dose management system 130, such as data obtained from the smart monitor device 120 regarding access events and their associated timestamps. The dose management application 112 can also be used and configured to receive management parameters from the dose management system 130 and, thereby facilitating management of product on the user device 110. And, by communicating with the smart monitor device 120, the dose management application 112 of the user device 110 can receive a token from the smart monitor device 120 and communicate that token to the dose management system 130.

In certain example embodiments, the dose management application 112 can also be used and configured to receive, from the smart monitor device 120 and via the communication application 111, refill data or other information that can be transmitted to the dose management system 130, thereby triggering a refill request as described herein. The dose management application 112 can also be used and configured to provide notices to the user regarding a missed dose or a connection error, such as via providing a text notification or other alert on a user interface of the user device 110. In certain example embodiments, the dose management application 112 can include a user interface (not shown) that allows a user 101 or operator of the dose management system 130, for example, to input information into the dose management application 112. For example, a user 101 or other system operator may use a user interface of the dose management application 112 to input user credentials into the dose management application 112, which can then be used to authenticate the user device 110 as described herein. The user interface of the dose management application 112 can also be used and configured to receive, via the communication application 111, data or information regarding the product to be managed and associated management parameters, such desired reminder times and/or dosage time interval data or information.

As shown in FIG. 1, the user device 110 includes a data storage unit 113 that is accessible by the communication application 111 and the dose management application 112. In certain example embodiments, the data storage unit 113 can, at the option of the user 101, store user credentials in a user device log 114. The data storage unit can also store a token log 115 that can include one or more tokens associated with one or more smart monitors 120. For example, to manage multiple products, the token log may store multiple tokens, either for the same user or for different users. In certain examples, the data storage unit 113 can store connection content related to the connection of the user device 110 to other network devices, as well as adherence data received from the smart monitor device 120. The exemplary data storage unit 113 can include one or more tangible computer-readable media. The data storage unit 113 can be stored on the user device 110 or can be logically coupled to the user device 110. For example, the data storage unit 113 can include on-board flash memory and/or one or more removable memory cards or removable flash memory.

The smart monitor device 120 represents the component of the exemplary operating system 100 responsible for monitoring access event data, receiving a refill request from the user device 110 (e.g., via a user input into the device, such as via a button-click user control option) and initiating the refill request, and/or providing audio and/or visual alerts to the user 101 regarding a processed refill or a possible missed dose. The smart monitor device 120 can include a dose monitoring module 121 that performs the analytical functions of the smart monitor device, including, for example, identifying an access event and associating the access event with a timestamp. The dose monitoring module 121 may also receive and process alert instructions that direct the smart monitor device 120 to provide one or more alerts, such as alerts at different times.

To provide an alert, the smart monitor device 120 can include an alert indicator 122 that operates to inform the user 101 of an event associated with management of the product. For example, upon receiving an instruction from the data management application 112 of the user device 110 to provide an alert for a missed dose, the dose monitoring module 121 can trigger the alert indicator to inform the user 101 of the possible missed dose. In another example embodiment, the dose monitoring module 121 may receive an instruction to notify the user 101 that a user-requested refill is ready, in which case the dose monitoring module 121 can trigger the alert indicator 122 to alert the user 101 regarding the refill. The alert provided by the alert indicator 122 can be any type of notification, such as an audio alert (e.g., a beep or buzz), a visual alert (e.g., a blinking light), or other detectable or perceptible alert (e.g., a vibration or haptic alert or notification).

In certain example embodiments, the smart monitor device 120 can include a clock or timer (not shown) that, for example, can be configured to trigger an event associated with the smart container 120 to occur at a certain time. The clock, for example, can be used to time-stamp events in real time and to manage alerts. For example, the clock or timer may trigger an alert at a specific time. And in certain example embodiments, the clock can be used to provide reminders to a user 101. For example, the smart monitor device 120 can, in certain example embodiments, provide reminders on its own via coordination with the clock or timer.

To communicate with other devices of the exemplary operating system environment 100, such as the user device 110, the smart monitor device 120 includes a short-range communication capability, such as Bluetooth, near field communication (“NFC”), a wireless short-range 2.4 GHz connection or any combination thereof or any other appropriate short-range architecture or system that facilitates the communication of signals, data, and/or messages to a computing device, such as the user device 110. Such short-range communication capabilities are beneficial, for example, as this provides a lower power burden, i.e., short-range transmissions use less power in comparison to longer-range transmissions. In certain example embodiments, the smart monitor device 120 can communicate directly with the network 105 and hence other devices on the network, such as via a low power cellular connection (e.g., NB-IoT) (dashed line, FIG. 1). In certain example embodiments, the short-range data communications between the user device 110 and the smart monitor device 120 are conducted at the 2.4 GHz wireless frequency, with an energy level provided by the electronic circuitry to provide a wireless transmission distance from the smart monitor device of between 50 and 1500 feet when transmitted in an open space, such as a range of between 50 and 300 feet. In certain example embodiments, a Bluetooth connection will be automatically established and held between the smart monitoring device 120 and the user device 110 whenever the two devices are in range of wireless communication, as according to protocols established in the Bluetooth 5.0 specification.

As shown in FIG. 1, the smart monitor device 120 includes a data storage unit 123 that is accessible, for example, by the dose monitoring module 121 and the communication module (not shown). The dosage storage unit 123 can include an access event log 124, such as a record of access events and associated timestamps for the access events associated with the smart monitor 120. The data storage unit 123 can also store a token, such as unique identifier for the smart monitor device 120 as described herein, the token being stored in a token storage 125. The exemplary data storage unit 123 can also include one or more tangible computer-readable media. The data storage unit 123 can be stored on the smart monitor device 110 or can be logically coupled to the smart monitor device 120. For example, the data storage unit 123 can include on-board flash memory and/or one or more removable memory cards or removable flash memory. In certain example embodiments, the data storage unit 123 may include between 100 and 5,000 kB of flash memory and between 24 and 1000 kB of RAM memory.

As will appreciated by those skilled in the art in view of this disclosure, the smart monitor device 120 described herein can be coupled to or otherwise associated with a container or dispenser, for example, such as a pill bottle or other receptacle. An example smart monitor device 120 that is compatible with the methods and systems described herein is disclosed in U.S. Pat. Pub. US20160048657, which is hereby expressly incorporated herein by reference in its entirety.

The dose management system 130 represents the component of the exemplary operating system 100 responsible for managing a product, such as in coordination with the user device 110, the smart monitor device 120, and the optional provider system 140. As shown in FIG. 1, the dose management system 130 includes a data processing module 131 that performs the analytical functions of the dose management system 130, such as authenticating the user device 110 of the user 101, managing a product associated with the user 101 (including a dose schedule for the product), processing refill requests received from the smart module 120, analyzing adherence data, verifying system connections, and/or triggering smart reminders.

As shown, the dose management system 130 can also include a web site 132 and an associated web server 133. The website 132, for example, can provide a user interface that can be accessed via the network 105. For example, a user 101, an operator (not shown) of the dose management system 130, and/or an operator of the provider system 140 (not shown) may access the dose management system 130 via the web site 132 to provide data and information to the dose management system 130, such as product characteristics for the product to be managed and/or credentials of the user 101. Such information can also include, for example, management parameters that can be input into the user interface of the website 132 and used to mage the product. The web server 133, for example, can operate to provide content to the website 132, such as features and user-interface content that the user 101, operator of the dose management system 130, and/or operator of the provider system 140 can use to input information to the dose management system 130, thereby permitting the dose management system 130 to manage a product for the user 101.

As shown, the dose management system 130 can include a data storage unit 134 that is accessible by the data processing module 131 and the web server 133, for example. The data storage unit 134 can store information regarding a user 101, such as a dose schedule for a given medication or other product that is to be managed. In certain example embodiments, the data storage unit 134 can, at the option of the user 101, store user credentials in a user log 135. The data storage unit can also store a token log 136 that can include one or more tokens associated with one or more smart monitors 120. The exemplary data storage unit 134 can also include one or more tangible computer-readable media. The data storage unit 134 can be stored on a device of the dose management system 130 or can be logically coupled to the a devoice of the dose management system 130. For example, the data storage unit 134 can include on-board flash memory and/or one or more removable memory cards or removable flash memory. In certain example embodiments, the dose management system 130 may be run on a series of distributed servers, sometimes referred to as “the cloud,” and may be managed by a third-party cloud services company, such as Microsoft Azure™ or Amazon™ Web Services.

In certain example embodiments, the exemplary operating system 100 can optionally include a provider system 140. The provider system 140, for example, represents a third-party system that can determine product characteristics and, via a communication application 143, for example, communicate the product characteristic to the dose management system 130, the user device 110, and/or the smart monitor device 120. For example, the provider system 140 may manufacture, supply, and/or provide access to one or more products, such as a medication or other product that can benefit from the product management methods and systems provided herein. The dose management module 141 of the provider system 140 can, for example, determine when a particular product should be taken or administered. The dose management module 141 can also determine dosage time interval information for the product and thereafter include such information with the management parameters. The dose management module 141 can also associate the product to be managed with a specific product identifier, such as a Universal Product Code (“UPC”) or, in the case of a medication, for example, and National Drug Code (“NDC”).

As shown, the provider system 140 can also include a web browser 142 that can be used to access the website 132 of the dose management system 130. For example, an operator (not shown) of the provider system 140 may use the web browser 142 to input information regarding the user 101 and/or the product characteristics into the dose management system 130 via the website 132 of the dose management system 130. The dose management system 130 can then use the received information according to methods and systems described herein. In certain examples, the provider system can also include a data storage unit (not shown), which can be used, for example, to store information regarding one or more products that may benefit from the product methods described herein. The data storage unit of the provider system 140 can be stored on a device of the of the provider system 140 or can be logically coupled to the a devoice of the of the provider system 140. For example, the data storage unit of the of the provider system 140 can include on-board flash memory and/or one or more removable memory cards or removable flash memory.

As those skilled in the art having the benefit of this disclosure will appreciate, all of the functions of the dose management system 130 can be performed on the user device 110, such as in conjunction with (or as integrated as part of) the dose management application 112. For example, the product characteristics and management parameters can be provided to the dose management application 112 of the user device 110, with the dose management application 112 thereafter managing the product as described herein. In certain other example embodiments, one or more of the functions of the dose management system 130 can be performed separately and independently from the user device 110 and vice versa. In other example embodiments, all or part of the functions of the dose management system 130 can be performed on the provider system 140, such as in conjunction with (or as integrated as part of) the dose management module 141 of the provider system 140.

In certain example embodiments, information can be communicated between the dose management system 130 and the provider system 140 over the network 105. For example, the provider system 140 may be an electronic health records system used by prescribing doctors to send prescribing information to the dose management system, and to receive medication adherence information from the dose management system 130 that can be displayed in the patient's personal electronic health record. Data generated by the dose management system 130 can be combined with information in the provider system 140 to calculate patient risk levels for medication non-adherence or other risk factors. Such patients may also be flagged for intervention by the healthcare provider.

It will be appreciated that the network connections shown in FIG. 1 are exemplary and that other means of establishing a communications link between the computers and devices can be used. Moreover, those having ordinary skill in the art having the benefit of the present disclosure will appreciate that the user device 110, the smart monitor device 120, the dose management system 130, and the provider system 140 illustrated in FIG. 1 can have any of several other suitable computer system configurations. For example, a user device 110 embodied as a mobile phone or handheld computer may or may not include all the components described herein.

### Example Processes

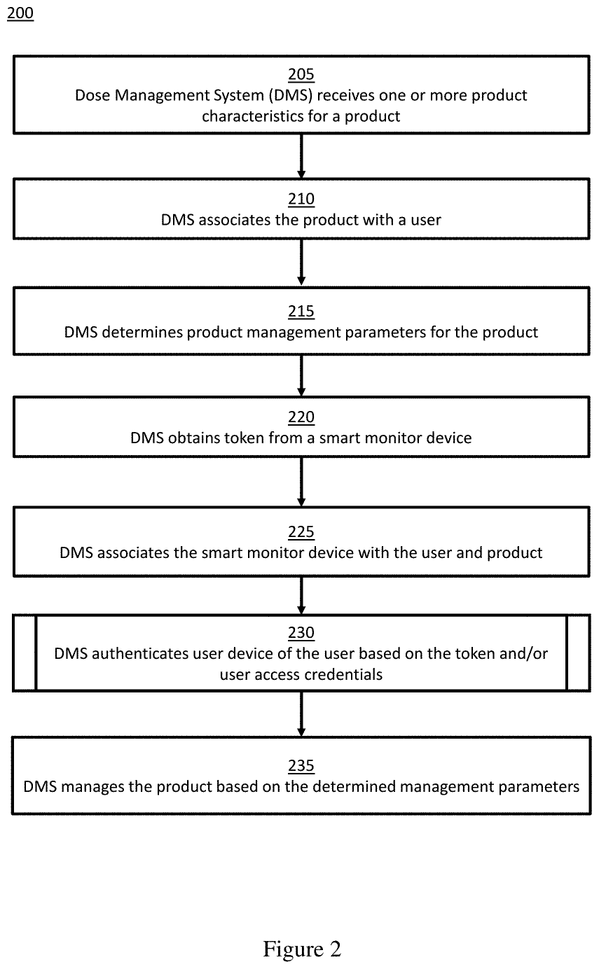

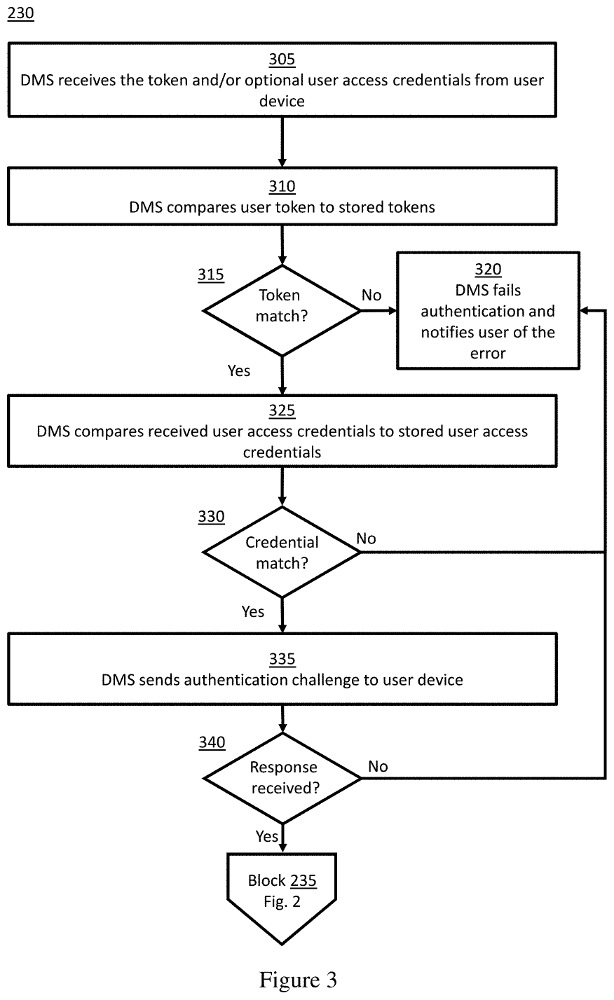

The components of the example operating environment 100 are described hereinafter with reference to the example methods illustrated in FIGS. 2-8.

FIG. 2 is a block flow diagram depicting a method 200 for enrolling a user and user device into a dosage management system 130, in accordance with certain example embodiments.

With reference to FIGS. 1 and 2, in block 205 of FIG. 2, the dose management system 130 receives one or more product characteristics for a product. That is, for a product that is to be managed, one or more features identifying a product and/or describing how the product is to be used, taken, and/or administered are provided to the dose management system 130. The product characteristics can include, for example, any information that identifies the product to be managed or that can be used to identify the product to be managed, such as the product name or product identifier (e.g., a Universal Product Code (“UPC”) or, in the case of a medication, for example, and National Drug Code (“NDC”)). The product characteristic can also include the quantity of the product provided to (or to be provided to) a user 101, how often a product dose is to be taken or administered (e.g., every 6 hours) or the time when the dose(s) are to be taken or administered (e.g., at 8 a.m. and 4 p.m. daily).

In certain example embodiments, the one or more product characteristic can include the amount of the product (eg., 25 mg or 100 ml) to be taken or administered at each specific time. If the product is a medication, for example, the one or more product characteristics can identify the specific medication, for example, and can indicate the user 101 is to take the medication three times of day, such as at morning, noon, and night. The one or more product characteristic may also include other instructions or information, such as that the medication may cause drowsiness and/or should be taken with food. If the product is pool chemical, for example, the one or more product characteristic can identify the specific pool chemical, as well as identify when require a user 101 is to add the pool chemicals to the pool (e.g., once per day or once per week).

Additionally or alternatively, the one or more product characteristics may specify the quantity of the product to be managed, without any information regarding when or how often the product should be taken or administered. For example, the product characteristics can identify a specific medication, the quantity of the medication (e.g., 30 doses), and that the medication can be taken on “as needed” basis. The one or more product characteristics can also specify how many refills of the product are available or that the user 101 may receive an indefinite number of refills. For a medication, for example, the one or more product characteristics may specify that, after the initial medication prescription is filled, only eleven refills are available. If the product is a laundry detergent, for example, the product characteristic can merely identify the specific detergent, such as by brand name. In certain example embodiments, the one or more product characteristics can also identify the amount of the detergent along with the recommended amount that is to be used with each load of laundry, such as 30 scoops and 1 scoop per load.

To receive the one or more product characteristics according to block 205, any system operator and/or user, such as an operator associated with a provider system 140, an operator associated with the dose management system 130, or a product manufacturer having access to the dose management system 130 can configure and determine product characteristics. For example, if the product is for a prescription medication, a physician at a provider system 140 (such as a doctor's office) can determine the one or more product characteristics for the medication. If the product is a pool chemical, for example, the chemical's manufacture can determine the one or more product characteristics for the medication for the pool chemicals. For example, the manufacturer can input the name of the pool chemical into the dosage management system 130 as described herein, as well as how many refills are available for the chemical. Additionally or alternatively, a licensed prescriber (e.g. a doctor) could enter the one or more product characteristics for the medication into the user interface of an electronic health record system for physicians, with the data from the electronic health record system syncing data over the network 105 to the dose management system 130.

In such example embodiments, the physician or manufacturer can use the web browser 142 of the provider system 140 to access the website 132 of the dosage management system 132, thus providing an interface for the physician or manufacturer to input the product characteristics into the dose management system 130. In certain example embodiments, the product characteristics can be communicated to the dose management system 130 via a secure connection over the network 105, such as secure connection (e.g., a virtual private network) between the communication application 142 of the provider system 140 and the communication module (not shown) of the dose management system 130. The dose management system 130 then receives the product characteristics. The dose management system 130 may also receive a product characteristic directly from a pharmacy management system (not shown) or an ePrescription software system (not shown).

In certain example embodiments, a user 101 may present a written prescription from a physician to a pharmacist, the prescription providing the one or more product characteristics for the medication. The pharmacist can then input the one or more product characteristics for the medication into the dose management system 130, such as by accessing the website 132 of the dose management system 130 and inputting the one or more product characteristics into a user interface of the website 132. Additionally or alternatively, the user 101 can communicate the one or more product characteristics to the dose management system 130, such as by inputting the one or more product characteristics content into the dose management application 112. The dose management application 112 can then communicate the one or more product characteristics to the dose management system 130, such as via the communication application 111 of the user device 110 and via the network 105. The dose management system 130 then receives the one or more product characteristics via the network 105.

In block 210, the dose management system 130 associates the product with the user 101. That is, once the dose management system 130 receives the one or more product characteristics, the data processing module 131 of the dose management system 130 reads the one or more product characteristics to identify the product and then assigns the product to a particular user 101. For example, if the product is the subject of a medication prescription, the data processing module 131 of the dose management system 130 receives the prescription and thereafter reads the prescription to identify the user 101 to which the prescription is directed. If the prescription is a blood pressure medication for Jane Doe, for example, then the data processing module 131 can read the prescription to identify Jane Doe as the user 101. In certain example embodiments, the dose management system 130 can also determine additional information about the user 101 from by reading the one or more product characteristics, such as the user's full name, address, telephone number, social security number, password, and/or other user credentials that may be associated with or included as part of the product characteristics. For example, a medication prescription received via the network 105 may contain such user credentials, along with the name of the medication being prescribed, the amount to be provided to the user 101, a dose schedule for the mediation, and/or the number of refills that are permitted.

Additionally or alternatively, a pharmacist may review a received prescription and, via one or more input selections on a user interface of the of the website 132 of the dose management system 130, search for the user 101. For example, the pharmacist may search for the user 101 based on the user's date of birth, phone number, and/or name. If the user 101 is already enrolled with the dose management system 130, then the pharmacist can select the user 101, such as from a drop-down list of users or user date-of-birth information, and assign the prescription to the user 101 in the dose management system 130, thereby associating the user 101 with the prescription. For example, the user may show as already existing in the dose management system 130. But if the user 101 is not already associated with the dose management system 130, the pharmacist, for example, can enter the user 101 information into the dose management system 130, such as via a user interface of the website 132 of the dose management system 130. The pharmacist can then input any additional user credential information, such as user's full name, date-of-birth, email address, home/residence address, telephone number, and/or other user credentials into the dose management system 130.

In certain example embodiments, once the dose management system 130 identifies the user 101 to which the identified product relates, the data processing module 131 of the dose management system 130 records the one or more product characteristics—along with the credentials of the user 101—in an accessible storage database, thereby associating the product to be managed with the user 101. For example, the user/product association can be recorded in the data storage unit 134 of the dose management system 130, such as in the user log 135 of the dose management system 130. If the user 101 is previously enrolled in the dose management system 130, for example, the data processing module 131 can record the received product characteristics is an entry in a user log 135 associated with the user 101. If the user 101 has not previously enrolled in the dose management system 130, the dose management system 130 can record any received user credential information in the user log 135, along with the received one or more product characteristics. The data processing module 131 can then later access the user log 135 as needed, such as via the data processing module 131, to manage the product as described herein.

In block 215, the dose management system 130 determines one or more product management parameters for the product. That is, the data processing module 131 of the dose management system 130 reads the received one or more product characteristics to identify any instructions or information that concern the management of the product (and that should be used to manage the product). For example, based on reading the product characteristics, the data processing module 131 can determine how often a refill of a product may be available. If the product to be managed is a medication, for example, the data processing module 131 can determine from the product characteristics that the medication is to be taken twice per day, morning and evening, with twelve refills being available (as an example). Hence, the data processing module 131 can determine—from the product characteristics—any instructions or information that can be used to manage the product, such as a dose schedule.

In certain example embodiments, and based on reading the one or more product characteristics, the data processing module 131 can determine, as one of the management parameters, one or more dosage time intervals to associate with the product. The dosage time interval, for example, refers to the window of time a scheduled dose of a product can be taken or administered such that scheduled dose is deemed to have been complied with. For example, if a medication prescription requires that requires a dose of the medication be taken at 9:00 a.m. and 6:00 p.m. each day, then the data processing module 131 may determine, as part of the management parameters, that the dosage time intervals are 7:00 a.m. to 11:00 a.m. for the 9:00 a.m. dose and 4:00 p.m. to 8:00 p.m. for the 6:00 p.m. dose, i.e., a dosage time interval of 4 hours for each dose.

In certain example embodiments, the data processing module 131 can dynamically split two scheduled doses based on the nearest dose time. For example, if two doses are scheduled to be taken at 9:00 am and 5:00 pm each day, a dose taken at 12:59 pm can be assigned to the 9:00 am dose while a dose taken at 1:01 pm can be assigned to the 5:00 pm dose. As those skilled in the art will appreciate based on this disclosure, the dose management system 130 can determine such dosage time intervals to be any interval that facilitates management of the product. Such dosage intervals can then be used as described herein to manage a product, such determining user adherence to a schedule of product doses and/or providing reminders to the user.

Additionally or alternatively, in certain example embodiments the management parameters can included parameters for scheduling reminders. For example, the data processing module 131 can determine that reminders—such as reminders to take or administer a product—should occur at a particular time, such as at or near a scheduled doses times identified in the one or more product characteristics. When the product is a prescription medication, for example, the dose management system 130 may determine that a reminder should occur within a dosage time interval of the scheduled dose for the medication.

In certain example embodiments, an operator of a provider system 140 may input one or more management parameters into the dose management system 130, such as via a user interface of the website 132. For a medication, for example, a prescribing physician may provide, as part of the product characteristics, a time window in which a dose of the medication should be taken, such as one or two hours before or after a scheduled dose. By reading the product characteristics, the data processing module 131 can then determine the preferred dosage time interval as a management parameter. And based on the dosage time interval, for example, the data processing module 131 can determine and configure reminders of the determined preferred dosage time interval as described herein.

Additionally or alternatively, the dose management system 130 may receive dose management parameters from the user 101. For example, if a user 101 wants to ensure that they add pool chemicals to a pool at a certain time of day, such as at noon each day, the user 101 can use an interface of the dose management application 112 to configure and input the management parameters for the desired dose schedule. The dose management application 112 can then communicate the user-initiated management parameters, such as via the communication application 111 and the network 105. The dose management system 130 can then receive the communication over the network 105. In this same or similar way, the user 101 can also provide desired reminder requests to the dose management system 130, thereby allowing the dose management system 130 to manage user-initiated reminders for the user 101. For example, the user 101 can use the dose management application 112 to configure one or more specific reminders times, and the dose management application 112 can communicate the reminder times to the dose management system 130. The data processing module 131 can then associate the dose management parameter with the user and thereafter use the management parameter to manage the product as describe herein to provide a reminder.

In certain example embodiments, once the dose management system 130 determines the management parameters for the product, the dose management system 130 can store the management parameters in an accessible storage database, such as in the data storage unit 134 of the dose management system 130. For example, to associate the management parameters with a specific user 101, the dose management system 130 can record the determined dose management parameters in the user log 135 for the user 101. Thereafter, the data processing module 131 can access the management parameters and thereby use the parameters to manage the product.

In block 220, the dose management system 130 obtains a token from a smart monitor device 120. That is, the dose management system 130 receives a token from the smart monitor device 120, the token being specific to the smart monitor device 120 and thus allowing identification of the smart monitor device 120. The token, for example, can be any grouping and/or series of numbers, letters, characters, digits, symbols, text or the like that provides a specific identifier for the smart monitor device 120 (i.e., a device-specific identifier). In certain example embodiments, the token can be a 12-character hexadecimal code, which follows the standard MAC address format. The token, for example, can be a unique hardware identifier that is written into an area of durable memory on the smart monitor device 120, such as in the token storage 125 of the data storage unit 123 of the smart monitor device 120. This can occur, for example, during the manufacturing process of the smart monitor device 120. The token, for example, can also be included as part of the programming of the smart monitor device 120, such as during manufacture of the smart monitor device 140, so that the token can be transmitted to another computing device that is capable of receiving the token. The token can be stored, for example, in the accessible token storage 125.

Additionally or alternatively, to improve security, in certain example embodiments the embedded hardware token can be periodically changed, so long as the registers of the operating environment 100 storing the token is updated. The token information can also be included in the packaging of the smart monitor device 140, such as on a matrix code or other readable label or insert associated with the packaging. And because the smart monitor device 120 can be associated with a container, for example, the token can be used to identify the container to which the smart monitor device 120 is attached.

The container can be any type of container, including, for example, a container for pills or tablets, a container for vitamins or supplements, an inhaler, a syringe, a container for liquid medication, a container for pool or water treatment chemicals, a container for industrial chemicals, a container for lab chemicals, a container for food or food ingredients, a container for aircraft or machine maintenance fluids, a cosmetic cream or lotion a container for facewash or shampoo, a container for mouthwash or toothpaste, an eye drop container, a nasal spray container, a pet food container, a baby food container, a cigarette container. The container can also include trash receptacles, mailboxes, cleaning supplies packaging, a container for cannabis products, a container for candy or breath mints, infant formula container, nutritional food packaging, water bottles, a container for coins or money, beverage containers, a container for gasoline, a container for holding additional interior packaging or a container for any other consumable good. In certain example embodiments, the smart monitoring device 120 could part of, or integrated within, any health monitoring device, such as a blood glucose meter, blood pressure cuff, an inhaler, a scale, a heart rate monitor, a blood alcohol reader, a hearing aid, smart eyeglasses, a smart watch, a thermometer.

To obtain the token from the smart monitor device 120, for example, an operator the dose management system 130 or a provider system 140 can scan a barcode or matrix code (e.g., a QR code) that is included with or attached to the smart monitor device 120. The content of the barcode or matrix code can correspond to the specific token of the smart monitor device 120. For example, the matrix code can include the same sequence of numbers, letters, digits, etc. of the token (and hence match the token included as part of the programming of the smart monitor device 120). Additionally or alternatively, an operator of the dose management system 130 or the provider system, for example, can input a code for the token into the dose management system 130, such as via the website 132 of the dose management system 130. In such example embodiments, the content of the code can correspond to or include the token for the smart monitor device 120. Additionally or alternatively, the smart monitor device 120 can communicate the token over a short-range field, such over Bluetooth, RFID, infrared, a decodable audio signal, and/or Near Field Communication, or other short-range means as described herein. A receiver (not shown) of the dose management system 130 or of the provider system 140 can then the detect the communication and read the content of the communication, thereby determining and receiving the token for the smart monitor device 120 from the communication.

In block 225, the dose management system 130 associates the smart monitor device 120 with the user 101 and the product. That is, to manage a product, the token from the smart monitor device 120 is assigned to the user 101 (and hence to the product associated with the user). For example, the token is recorded with the dose management system 130 such that locating and retrieving the token also identifies the user 101. For example, if the product is a medication and a pharmacist is filling a prescription for the medication, the pharmacist can fill a standard pill container with the prescribed amount of medication. The pharmacist can then attach a smart monitor device, such as smart pill cap (see U.S. Pat. Pub. US20160048657), to the pill bottle. The pill bottle including the medication can then be provided to the user 101. If the product is a pool chemical, for example, the manufacturer of the chemicals can use a smart lid, for example, that includes the smart monitor device 120. The container including the smart lid can then be provided to the user 101. In such examples, the token from the smart monitor device 120 can be recorded with the dose management system 130, along with an entry that identifies the user 101, thereby associating the smart monitor device 120 with the user 101.

In certain example embodiments, the token can be recorded in an accessible data storage unit 134 of the dose management system 130. For example, the token can be recorded in a token log 136 along with information identifying the user 101 associated with the token. Additionally or alternatively, the token can be recorded with the user information in the credential log 135 of the user 101. The dose management system 130 can then access the token log 136 as described herein to manage the product for the user 101, such as when authenticating a user device 110 of the user 101. Additionally or alternatively, the received token can be recorded in the user log 135, such as in an entry of the user log 135 that also identifies the user 101. Hence, by associating the token with the user 101, retrieval of the stored token can identify the user 101 and vice versa (i.e., retrieval if of the user identity can retrieve the token).

In block 230, the dose management system 130 authenticates a user device 110 of the user 101 based on the token and/or the user access credentials. That is, the dose management system 130 receives the token from the user device 110, reads the received token, and compares the received token to the token log 136 to identify a token match (i.e., a match between the token recorded with the dose management system 130 and the received token). The dose management system 130 also optionally receives user credentials from the user device 110 and compares the credentials to the credential log 135 to identify a credential match. Based on one or more of the matches, the dose management system 130 authenticates the user device 110 of the user 101. And in certain example embodiments, the dose management system 130 can provide an authentication challenge to the user 101, thereby further authenticated the user device. The details of block 230 are described in further detail below with reference to FIG. 3.

In block 235 of FIG. 2, once the user device 110 of the user 101 has been authenticated, the dose management system 130 manages the product for the user based on the determined management parameters. That is, following user device authentication as described herein, the user device 110, the smart monitor device 120, the dose management system 130, and the provider system 140 can stay in communication with each other, such as over the network 105. The data processing module 131 can then retrieve the management parameters as needed and apply the parameters to manage the product. For example, in certain example embodiments the dose management system 130 can process a refill request for the user 101, such as by comparing a refill request to management parameter that identifies the number of refills that remain available for the product.

Additionally or alternatively, the dose management system 130 can monitor adherence of the user 101 to a dose schedule for the product, such as by relying on dose management parameters that define which a dose of a medication, for example, should be taken or administered. The data processing module 131 can also, by comparing obtained user adherence data to one or more management parameters that provide dosage time intervals for the product, provide intelligent reminders for a user 101 take or administer the product. The data processing module 131 can also rely on refill management parameters, for example, to provide automatic refills for the user 101. The and other methods and embodiments are described in further detail below.

FIG. 3 is a block flow diagram depicting a method 230 for authenticating a user device 110 for management of a product, in accordance with certain example embodiments.

With reference to FIGS. 1-3, in block 305 of FIG. 3, the dose management system 130 receives the token and/or user access credentials from user device 110. For example, as part of the user's enrollment with the dose management system 130, the user 101 installs the dose management application 112 on the user device 110, thus facilitating communication between the user device 110 and the smart monitor device 120. In certain example embodiments, the dose management system 130 may take steps to facilitate such enrollment, such as sending the user a text message or email with a link to download the dose management application 112 upon enrollment of the user into the dose management system 130. In such example embodiments, the smart monitor device 120, which can be included as part of a container or receptacle as described herein, for example, can broadcast or otherwise communicate the token user device 110. For example, the smart monitor device 120 can advertise or broadcast the token over a short-range signal as described herein, and the user device 110 can receive the token.

Additionally or alternatively, the user 101 may press a button or activate a switch (or other mechanism) on the smart container 120, thereby causing the smart container 120 to advertise or broadcast the token. Based on the broadcast token, the user device 110 receives the token. The dose management application 112, for example, then communicates the token to the dose management system 130, such as via the communication application 111 and the network 105. The dose management system 130 then receives the token via the network 105.

With regard to the user access credentials, in certain example embodiments—and as part of installing the dose management application 112—the user 101 can input one or more user credentials into a user interface (not shown) of the dose management application 112 of the user device 110. For example, by inputting user credentials, the user 101 can create a user account associated with the dose management application 112 and the dose management system 130. Once the dose management application 112 receives one or more credentials of the user 101, the dose management application 112 can communicate the credentials to the dose management system 130, such as via the communication application 111 and the network 105. The dose management system 130 then receives the one or more user credentials via the network 105. The user credentials can include, for example, the user's name, date-of-birth, username, phone number, social security number, etc. or any other user-specific information.

In certain example embodiments, the received token and/or the received user credentials can be recorded in an accessible data base of the user device 110, such as the data storage unit 123. For example, the received token and/or the received user credentials can be recorded in the token log 115 and/or the user device log 114 of the user device 110. The dose management application 112, for example, can then access the token information of the user 101 and/or the user credential information as needed in order to execute the methods described herein. In certain example embodiments, the stored token and/or credentials can be used to later re-authenticate the use device 110 as described herein. In certain example embodiments, the stored credentials of the user 101 can be used to keep the user 101 logged into the dose management application 112 or to re-log the user 101 into the dose management application 112. The stored credentials of the user 101 on the user device 110 can also be used, for example, to process a refill request for the user 101 as described herein.

In block 310, the dose management system 130 compares the received token to the stored token log 136. That is, the data processing module 131 of the dose management system 130 compares the received (second) token—i.e., the token received from the user device 110 as described in block 305—to the token log 136 in an attempt to identify a match to the recorded token (i.e., the first token) received in block 220. To compare the tokens, for example, the data processing module 131 of the dose management system 130 reads the token received from the user device 110. The data processing module 131 can then access and read the token log to identify a token match between the stored first token and the received second token. Based on reading the received token and the token log, the data processing module 131 can compare the numbers, letters, characters, digits, symbols, text or the like of the received (second) token with the numbers, letters, characters, digits, symbols, text or the like of one or more recorded (first) tokens recorded in the token log 136. A token match exists, for example, when a recorded (first) token matches the received (second) token.

If the dose management system 130 determines that a token match does not exist between a recorded (first) token on the token log 136 and the received (second) token, then the method follows the “NO” branch of block 315 to block 320 of FIG. 3. But if the dose management system 130 determines that a token match does exist between a recorded (first) token of the token log 136 and the received (second) token, then the method follows the “YES” branch of block 315 to block 325 of FIG. 3, in accordance with certain example embodiments.

In block 320, if the dose management system 130 does not identify a match between the received token and a recorded token of the token log, the dose management system 130 fails the authentication and, in certain example embodiments, notifies the user 101 of the authentication error. That is, based on an absence of the token match, the data processing module 131 of the dose management system 130 can determine that the smart monitor device 120 from which the received token was obtained does not match a token that was previously received and hence that the smart monitor device 120 has not been associated with the user 101, such as described in block 225 of FIG. 2. With the failed authentication, for example, in certain example embodiments the dose management system 130 will not fill a refill request or send a smart reminder, as described herein.

In certain example embodiments, based on the failed authentication, the dose management system 130 notifies the user 101 of the authentication error. Such notification can occur by any method that informs the user 101 of the error. For example, the dose management system 130 can communicate a message to the user device 110, such as a text message notifying the user of the failed authentication. The dose management system 130 can also trigger a telephone call to the user device 110 or other number associated with the user credentials, thereby informing the user 101 of the failed authentication. In certain example embodiments the data management application on the user device 110 can provide the alert to the user 101, such as by triggering an audio/visual alert on the user device 110.

Additionally or alternatively, in the absence of the authentication, the smart monitor device 120 can provide an alert to the user. For example, with a successful authentication, an indicator light on the smart monitor device 120 may blink red and then change to green once authentication occurs. Hence, a red light that does not change to green can indicate a failed authentication. Additionally or alternatively, the dose management system 130 can communicate an instruction to the user device 110 via the network 105, the instruction causing the user device 110 to trigger an alert on the smart monitor device 120. For example, in response to such an instruction, an indicator light on the smart monitor device 120 may blink or display a certain color indicating the failed authentication (and hence the failed enrollment of the smart monitor device 120 with the dose management system 130). Based on the failed authentication, the user 101, for example, can take steps to rectify the error, such as contacting an operator of the dose management system 130.

In block 325, if the dose management system 130 identifies a match between the received token and a recorded token of the token log, in certain example embodiments the dose management system 130 can compare the received user access credentials to stored user access credentials. That is, the data processing module 131 of the dose management system 130 can compare one or more of the received user access credentials received during the dose management application 112 installation with one or more user credentials recorded in the user log 135. For example, the data processing module 131 of the dose management system 130 can read the user credentials received in block 305 to identify a telephone number of the user 101. The data processing module 131 can then access and read the user log 135 for a matching telephone number. Based on reading the received user credentials and the stored user credentials, the dose management system 130, such as via the data processing module 131, can compare the credentials. For example, the data processing module 131 may identify a user credential match to a credential in the user log 135, thereby further authenticating the user device 110 of the user. Or, the data processing module 131 of the dose management system 130 may not identify a credential match between the received credentials and the received credentials, thereby prompting an authentication error.

If the dose management system 130 determines that a user credential match does not exist between the one or more received credential and one or more stored credentials in the user log 135, the method follows the “NO” branch of block 330 to block 320 of FIG. 3. That is, in the absence of a credential match, the dose management system 130 fails the authentication and optionally notifies the user 101 as described in block 320. But if the dose management system 130 determines that a credential match does exist between the one or more received credential and one or more stored credentials in the user log 135, then the method follows the “YES” branch of block branch of the block 330 of FIG. 3 to block 335.

In block 335, when the dose management system 130 identifies a credential match, in certain example embodiments the dose management system 130 sends an authentication challenge to the user device 110. That is, based on reading the received user credentials and identifying the credential match in the credential log, the data processing module 131 can retrieve, for example, a telephone number for the user 101 and/or an email address for the user 101 from the user credentials recorded in the user log 135. The data processing module 131 can then a provide authentication challenge to the user device 110 that is known to be associated with the user 101. For example, the data processing module 131 can send a text message or email to the user device 110 via the network 105. The authentication challenge, for example, can be a passcode, pin number, a deep link, a URL-contained credential, a CAPTCHA challenge, etc. to which the user 101 can respond via the user device 110. The user 101, for example, can respond to the challenge, for example, by inputting the pin number or code into a user interface of the user device. For example, the user 101 may respond to a text message by entering the received pin code into the responsive text message.

In block 340, if the dose management system 130 does not receive a response to the credential challenge, the method follows the “NO” branch of block 340 to block 320 of FIG. 3. That is, in the absence of a response from the user 101, the dose management system 130 fails the authentication and optionally notifies the user 101 as described in block 320. But if the dose management system 130 receives a response from the user 101 in response to the authentication challenge, then the method follows the “YES” branch of block 340 and returns to block 235 of FIG. 2. That is, based on the authentication, the dose management system 130 manages the product based on the determined management parameters.

As those skilled in the art will appreciate based on this disclosure, one or both authentication methods—i.e., authentication based on a token match and authentication based on user credentials—can be used to authenticate a user device 110 of the user 101 associated with the dose management system 130. Further, providing a challenge request can be used to further authenticate the user device 110 of the user 101. For example, the dose management system 130 can be configured such that each authentication method is used when a more secure authentication is desired. Also, it is to be understood that the authentication methods can occur in a different order. For example, the dose management system 130 may authenticate a user device 110 based first on a user credential match (and an authentication challenge) and thereafter a token match. Hence, provided are single-factor, two-factor, and multi-factor authentication processes.

Additionally, while the authentication methods described herein can be used to initially to authenticate a user device 110 associated with particular smart monitor device 120, one or more of the authentication methods or steps can be repeated at any point that re-authentication may be needed. For example, after an initial authentication, the dose management system 130 can be configured to re-authenticate the user device 110 at any step of processing a refill request. Additionally or alternatively, the dose management system 130 can re-authenticate the user device 110 on an hourly, daily, or weekly basis.

Additionally or alternatively, the dose management system 130 can re-authenticate the user device 110 each time the user device 110 and the smart monitor device 120 re-connect. For example, if the smart monitor device 120 and the user device 110 communicate over a Bluetooth connection, and the user device 110 later moves out of range of the smart monitor device 120, re-authentication can occur once the user device 110 is brought back in to range of the smart monitor device 120. Additionally or alternatively, the dose management system 130 may re-authenticate the user device 110 each time the user 101 logs out of the dose management application 112 and thereafter logs back in to the dose management application 112. For example, the user device 110 can remain authenticated while the user 101 is logged into the user device, only to require re-authentication when the user 101 logs out of the dose management application 112 and then attempts to log back in to the dose management application 112.

In certain examples embodiments, in order to keep a user device 110 enrolled with the dose management system 130 after authentication, the dose management system 130 can provide an authentication key to the user device 110. For example, once the data processing module 131 of the dose management system 130 authenticates the user device 110, the data processing module 131 can communicate an authentication key, such as an encrypted key code, to the user device 110 via the network 105. The user device 110 then stores the authentication key, for example, in the data storage unit 113 of the user device 110. The data processing module 131 can also record a copy of the key with the dose management system 130, such as in the user log 135 and/or token log 136 (and with the entries associated with the user 101).

Thereafter, the user device 110 can communicate the authentication key to the dose management system 130 via the network 105 as needed, and the dose management system 130 can receive the authentication key via the network 105. For example, if the user device 110 disconnects from the network 105 and then later reconnects, the user device 110 can communicate the authentication key to the dose management system 130 via the network 105. When the dose management system 130 receives the key, the data processing module 131 can then compare the received authentication key to any stored authentication keys, such as by reading the received authentication key and any stored authentication keys. If a match is determined between the received authentication key and a stored authentication key, then that user device 110 can be re-authenticated, such as without the user 101 having to take any additional steps. For example, the user 101 would not have to respond to an authentication challenge to re-authenticate the user device 110.

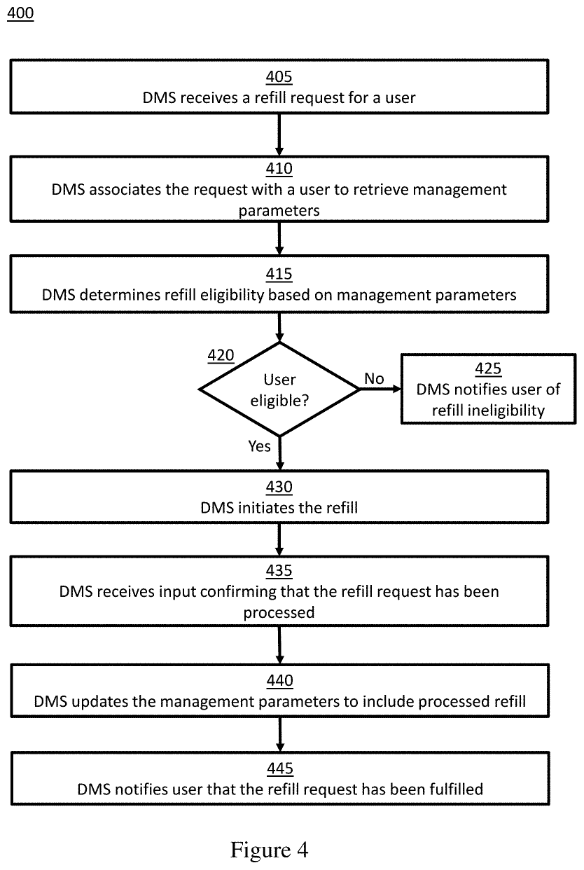

FIG. 4 is a block flow diagram depicting a method 400 for receiving and processing a refill request, in accordance with certain example embodiments.

With reference to FIGS. 1 and 4, in block 405 of FIG. 4, the dose management system 130 receives a refill request. That is, a user 101 provides an input to the smart monitor device 120 that initiates the refill request, and the request is communicated to the dose management system 130 via user device 110 over the network 105. For example, the user 101 may push a button or otherwise activate a switch or other user control option associated with the smart monitor device 120. The user input, for example, can cause the smart monitor device 120 to communicate a signal to the user device 110, such as over short-range signal as described herein. In certain example embodiments, the signal for the refill request can include the token, thereby facilitating identification and/or re-authentication of the user device 110 as described herein. In certain example embodiments, a refill request may consist of multiple button presses that are performed within a predefined unit of time, such as between 0.3 seconds and 3 seconds.

In certain example embodiments, the user control option for receiving the user input may not be built directly into a container of physically connected thereto. For example, the user control option for receiving a user input, such as a refill request, can remotely connect with the smart monitor device 120 and/or the user device 110 via a short-range connection, such as a 2.4 GHz connection. In such example embodiments, the remote smart button, for example, can provide similar functionality of providing for more convenient refills and optionally, for tracking the consumption of products, as described herein via the reading and analysis of adherence data. In certain example embodiments, such a remote user control options may include or be associated with an accessible memory, such that refill requests can be stored in memory if the smart button is out of wireless range of the user device 110 and/or the smart monitor device 120, such that refill request will be made upon connection by the smart button to the user device.

Once the user device 110 of the user 101 is enrolled in with the dose management system 130, the user device 110 can then receive the signal for the refill request from the smart monitor device 120 over a short-range connection. For example, the dose management application 112 can receive the signal for the refill request via the communication application 111. Thereafter, in certain examples embodiments the dose management application 112 can read the signal to determine that the signal is a refill request. The dose management application 112 can then associate with the request any information that may be beneficial in processing the refill request. For example, based on the user's login credentials that the user employed to access the dose management application 112, the dose management application 112 can associate one or more of the user access credentials with the refill request. In certain example embodiments, the dose management application 112 can associate the user's name with the refill request. In the example of a pharmacy refill, the refill request may include one or more of the user's name, date of birth, gender, payment type, insurance cardholder ID, Insurance RxGroup, Insurance Rx BIN, PCN, medication name, medication quantity to be dispensed, days supply, shipping method, shipping address, National Drug Code (NDC), name of the drug manufacturer, prescribing doctor's name, prescribing doctor's National Provider Identifier (NPI), pharmacy store number, pharmacy name, pharmacy identifier, or any other information required to send the refill.

Additionally or alternatively, the dose management application 112 can associate the token with the refill request. The dose management application 112 can then communicate the refill request and any associated information, such as user credentials and/or the token, to the dose management system 130 via the network 105. The dose management system 130 can then receive the refill request, such as via the network 105 and the communication module (not shown) of the dose management system 130. The dose management system 130 can then process the refill request.

In block 410, the dose management system 130 associates the refill request with the user 101 to retrieve the management parameters. That is, the data processing module 131 of the dose management system 130 identifies the user 101 to which the refill request pertains. To identify the user 101 and associate the user 101 with the refill request, the data processing module 131 of the dose management system 130 reads the refill request, including any user credential information and/or token associated with the request. In certain example embodiments, the data processing module 131 of the dose management system 130 can determine, based on reading the refill request and any associated information, the name of the user 101 to which the request pertains. The data processing module 131 can then, based on the user's name, retrieve the management parameters associated with the user 101 from the data storage unit 134 of the dose management system 130, thereby associating the refill request with the user 101. For example, the data processing module 131 reads the user credential log to identify the user 101 to which the refill request pertains. The data processing module 131 then identifies the user 101 and, based on the user identification, retrieves the one or more management parameters, such as the one or more management parameters relevant to managing a product refill. For example, the retrieved management parameters may identify the number of refills available for the user 101 and the product being managed.

Additionally or alternatively, the dose management system 130 can rely on other user credential information to retrieve the management parameters from the data storage unit 134, such as the user's telephone number or any other user-identifying information associated with the refill request. For example, the data processing module 131 of the dose management system 130 can compare a received user telephone number with telephone numbers stored in the user log 135, thereby permitting the data processing module 131 to identify the user 101 to which the refill request pertains. Additionally or alternatively, if a token is associated with the refill request, then the dose management system 130 can identify the user 101 (and hence retrieve the product management parameters) based on the received token. For example, the data processing module 131 of the dose management system 130 can use the token to identify the token associated with the user in the token log 136.

In block 415, the dose management system 130 determines the refill eligibility of the user 101 based on the management parameters. That is, once the data processing module 131 of the dose management system 130 associates the refill request with the user 101 to which the request pertains, the data processing module 131 confirms that the user 101 is in fact eligible for a refill before further processing the refill request. For example, based on identifying the user 101 and associating the user 101 with the refill request, the dose management system 130 reads the retrieved management parameters, such as those determined for the user 101 in block 215 of FIG. 2. The data processing module 131 of the dosage management system 130 then determines, for example, if the management parameters include refill information. The data processing module 131 can also determine if the allotted number of refills has been processed, such as by reading and identifying any refills previously recorded in the user log 135 and/or token log 136.

For example, if the product being managed is a medication having 3 refills, the dose management system 130 can read the management parameters for the medication to determine whether only one or two refills have been processed or whether the dose management system 130 has processed all three of the available refills. If all three refills have been processed, for example, then the data processing module 131 can determine that no refills are available. Conversely, if only two of three refills have been processed, the data processing module 131 can determine that a refill remains (i.e., that the user is eligible for a refill). Alternatively, the dose management system 130 may communicate the refill request to a third-party system to determine refill eligibility, such as a provider system 140, a pharmacy management system, or an ePrescription routing software system.

If the dose management system 130 determines that the user 101 is not eligible for a refill, then the method follows the “NO” branch of block 420 to block 425 of FIG. 4. But if the dose management system 130 determines that the user 101 is eligible for a refill, then then the method follows the “YES” branch of block 420 to block 430 of FIG. 4, in accordance with certain example embodiments.

Based on determining that the user 101 is not eligible for a refill, in block 425 the dose management system 130 notifies the user 101 of the refill ineligibility. That is, the data processing module 131 of the dose management system 130 can inform the user 101 that the refill was denied. Such notification can occur by any method that informs the user 101 of the refill ineligibility. For example, the data processing module 131 of the dose management system 130 can communicate a message to the user device 110, such as a text message notifying the user 101 that the refill request was denied. The dose management system 130 can also trigger a telephone call to the user device 110 or other number associated with the user credentials, thereby informing the user 101 of the refill ineligibility.

Additionally or alternatively, the dose management system 130 can communicate instructions to the smart monitor device 120 via the authenticated user device 110 and the network 105, the instructions causing an alert on the smart monitor device 120 indicating the that the refill request was not successful. For example, an indicator light on the smart monitor device 120 may blink or display a certain color indicating that the user 101 is ineligible for a requested refill. Based on the refill ineligibility, for example, the user 101 can take steps to address the lack of a refill, such as by contacting an operator of the dose management system 130 or provider system 140. In the case of a medication, for example, the user 101 can contract their pharmacist or healthcare provider.

Additionally or alternatively, the dose management system 130 can communicate the refill request to the provider system 140, with an indication that the user 101 is not eligible for a refill. For example, the data processing module 131 of the dose management system 130 can, via the network 105, communicate to the provider system 140 that the user 101 has requested a refill but that the user 101 is not eligible for a refill. The dose management module 141 can then receive the request via the network 105 and process the request. For example, the dose management module 141 can, in certain example embodiments, modify the management parameters to allow one or more additional refills, and thereafter communicate the modified management parameters to the dose management system 130 via the network. The data processing module 131 of the dose management system 130 can then retrieve and process the management parameters as described herein to initiate a refill of product.

In block 430, if the user 101 is eligible for a refill, then, in certain example embodiments, the dose management system 130 initiates the refill. That is, the dose management system 130 notifies an operator of the dose management system 130 and/or the provider system 140 that the product needs to be refilled. If the product is a medication, for example, the data processing module 131 can communicate to the user's pharmacist or other healthcare provider associated with the provider system 140, for example, that the user 101 is eligible for a requested prescription refill. For example, the data processing module 131 may trigger an audible and/or visual alert on a user interface of the dose management system 130 and/or provider system 140, the alert notifying the operator of the user-eligible refill request. The operator can then fulfill the request. For example, if the product is a medication, an operator of the provider system 140 can refill the medication. Likewise, if the product is a pool chemical the operator can retrieve the chemical and have the chemical ready for pickup (or ship the chemical to the user 101).

In certain example embodiments, the dose management system 130 can interface with an automatic refill system (not shown), such as via an application programming interface of an automatic refill system. In such a configuration, the dose management system 130 can communicate the refill request (and the user's eligibility) to the automatic refill system, such as via the network 105. The dose management system 130 can, in certain example embodiments, also communicate certain user credentials to the automatic refill system, such as the user's name, billing information, and/or shipping information. The automatic refill system can then process the user's refill request without, for example, involving an operator of the dose management system 130. For example, the automatic refill system can initiate an order for the product being refilled, such as at a third-party merchant. The merchant can then ship the order to the user 101 based on the user's credentials, thereby fulfilling the refill request.

In block 435, in certain example embodiments the dose management system 130 receives an input confirming that the refill request has been processed. That is, in certain example embodiments, once a user's refill request has been processed and the refilled product is ready for pickup or shipment, for example, the dose management system 130 is notified that the refill request of the user 101 has been fulfilled. For example, if a pharmacist refills a prescription medication based on the refill request, then the pharmacist can input a refill confirmation into the dose management system 130, such as via the website 132 of the dose management system 130. The dose management system 130 then receives the refill confirmation via the input.

In block 440, the dose management system 130 updates the management parameters to include the processed refill. That is, in certain example embodiments, in order to track the number of refills that have been processed—and therefore to later determine future eligibility of the user 101 to receive additional refills—the data processing module 131 modifies the management parameters to include an indication that a refill was processed. For example, once the refill confirmation from block 435 above is received, the data processing module 131 can record the refill confirmation in a log associated with the user 101, such as the user log 135 and/or the token log 136 as a management parameter for the product. Thereafter, when the data processing module 131 processes a future refill request, the refill can be counted against the number of refills remaining when the data processing module 131 reads the update management parameters. For example, if the dose management system 130 is managing a medication prescription having three refills, recording a first fulfilled refill confirmation permits the data processing module 131 to later determine—for the next refill request—that the user 101 has two refills remaining. Hence, by updating the product management parameters for the product being managed, the dose management system 130 can keep track of the number of refills remaining and hence manage refill requests.

In block 445, in certain example embodiments the dose management system 130 notifies the user 101 that the refill request has been fulfilled. That is, in certain example embodiments, the dose management system 130 communicates to the user 101 that the refill is ready for pickup and/or that a product refill has been shipped. For example, based on receiving the refill confirmation, the data processing module 131 of the dose management system 130 can communicate an instruction to the user device 110 of the user 101, thereby triggering the user device 110 to provide an alert to the user 101. For example, the dose management system 130 may send a text message or other smartphone notification to the user device 110 over the network 105, the text message communicating to the user 101 that the refill request has been fulfilled.

Additionally or alternatively, the dose management system 130 can communicate instructions to the smart monitor device 120 via the authenticated user device 110 and the network 105, the instructions causing an alert on the smart monitor device 120 indicating the that the refill request has been fulfilled. For example, an indicator light on the smart monitor device 120 may blink or display a certain color indicating that the refill request has been fulfilled. The user 101 can then take whatever action is needed to receive the refilled product. For example, if the refill request pertains to a medication refill, the user 101 can go to their pharmacist to pick up the refilled medication. Or, with the alert, the user 101 can be notified that the medication refill has been shipped to the user 101.

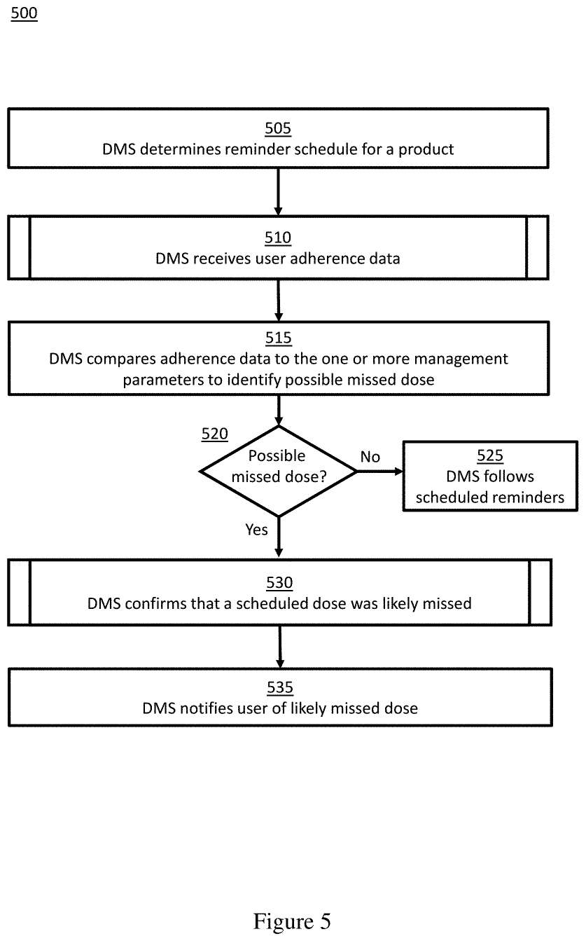

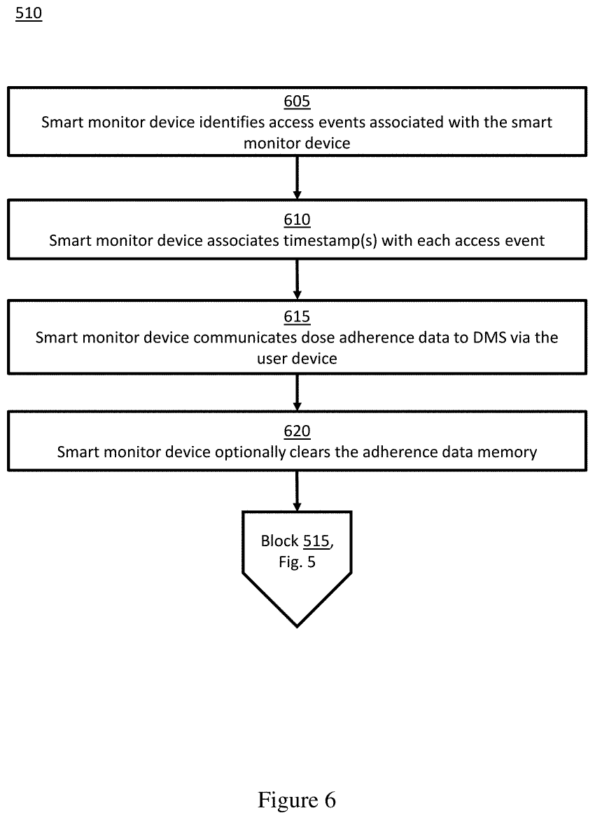

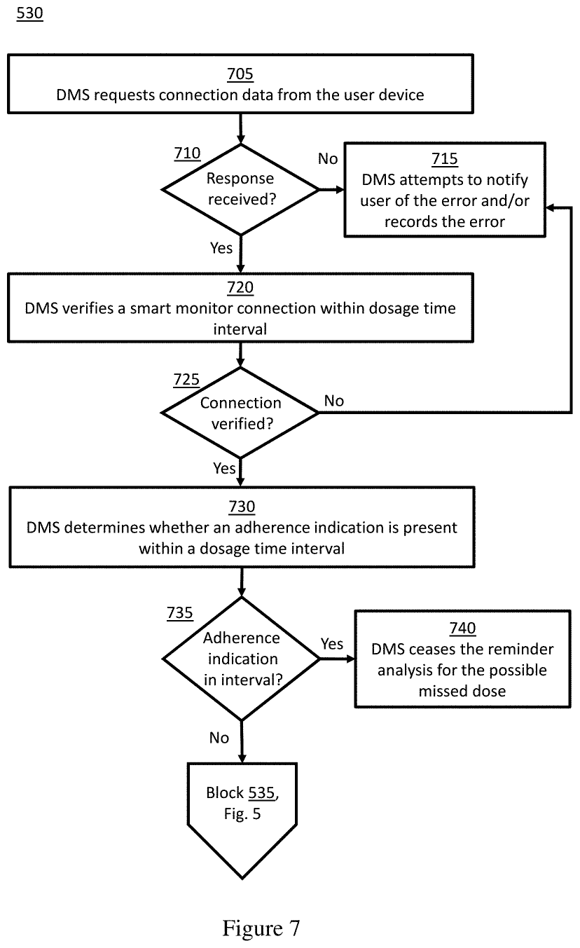

FIG. 5 is a block flow diagram depicting a method 500 for monitoring user adherence to a dose schedule, in accordance with certain example embodiments.

With reference to FIGS. 1-2 and 5, in block 505 of FIG. 5, the dose management system 130 determines a reminder schedule for a product. That is, for the product being managed, the data processing module 131 of the dose management system 130 can determine one or more times at which a reminder regarding a likely missed dosage should occur, based on a dose schedule for the product. For example, the data processing module 131 can read the management parameters for a product to determine if the product needs to follow a dose schedule, i.e., a specific set of times that the product should be taken or administered. The data processing module 131 can the determine and set reminders based on the dose schedule. For example, if a dose is scheduled at 9:00 a.m. each day, the data processing module 131 may schedule a reminder before or after the scheduled dose. The dose schedule, for example, can relate to any product as described herein, such a prescription medication, a non-prescription medication, a dietary supplement, a consumable cosmetic product, a consumable beverage, a consumable food product, or a non-consumable product.

In certain example embodiments, the reminders can be determined as part of the dose management parameters that are determined in block 215 of FIG. 2. For example, when determining the dose management parameters, the data processing module 131 can determine that a reminder is to be sent to the user 101 at any time after a likely missed dose is determined, such as at 1, 2, 3, 4, 5, 6, 7, 8, 9, 10, 11, or 12 hours after a likely missed dose. In certain example embodiments, the data processing module 131 can determine multiple reminders, such as a daily reminder to take or administer a dose of the product being managed. If the product is a medication, for example, the data processing module 131 may configure the reminder to occur within an hour after a likely missed dose of the medication is determined. In certain example embodiments, the data processing module 131 may configure a reminder to occur before a dose is to be taken or administered, such as at 1, 2, 3, 4, 5, 6, 7, 8, 9, 10, 11, or 12 hours before a dose is to be taken.

In certain example embodiments, such as when reminders are managed on the smart monitor device 120, the data processing module 131 communicates the determined reminder to the user device 110 via the network 105. The user device 110 can then communicate the received the reminder schedule to the smart monitor device 120 via a short-range connection as described herein. The smart monitor device 120 then receives the reminder schedule and can, in such example embodiments, manage the reminder scheduled as described herein. For example, the smart monitor device 120 can provide alerts to the user 101 to take or administer a product, without relying on the user device 110 or the dose management system 130. For example, when the smart monitor device 120 is associated with a container that includes a medication, the smart monitor device 120 can provide alerts to the user to take the medication.

In block 510, the dose management system 130 receives user adherence data. That is, the dose management system 130 receives data from the user device 110, the data including time-stamped access event entries. For example, the smart monitor device 120 determines access events associated with the smart monitor device 120. The access events can include, for example, time-stamped open/close events where the user 101 has opened a container including the smart monitor device 120 and then replaced the smart monitor device 120 on the container. The smart monitor device 120 then communicates the access event information to the user device 110 via a short-range connection, and the user device 110 receives the communication. The user device 110 then communicates the access event information to the dose management system 130 via the network 105. The dose management system 130 then receives the access event information as the adherence data, via the network 105. The details of block 510 are described in further detail below with reference to FIG. 6.

Turning to FIG. 6, provided is a block flow diagram depicting a method 510 for receiving user adherence data, in accordance with certain example embodiments.

With reference to FIGS. 1, 5, and 6, in block 605 of FIG. 6, the smart monitor device 120 identifies access events associated with the smart monitor device 120. That is, the data monitoring module 121 of the smart monitor device 120 identifies each access event associated with the smart monitor device 120, such as an open event and a close event of a cap including the smart monitor device 120. If the product being managed is a medication inhaler, for example, the data monitoring module 121—when coupled to the inhaler—can determine when the inhalant was discharged, the discharge corresponding to an access event for the smart monitor device 120.

In certain example embodiments, the access event may correspond to the opening of a blister pack and hence access to each pill, for example, included behind the blister wrap. If the product is a traditional pill-form medication, for example, the access event can be the opening of the pill bottle. A second access event can correspond to the closing of the pill bottle. If the product is a salve or ointment, for example, a first access event can correspond to the opening of the cap while a second access event can correspond to the closing of the cap onto the ointment or salve container. Hence, the access event can be any monitorable access to a container associated with the smart monitor device 120. If the product being managed is laundry detergent, for example, opening and/or closing a lid including the smart monitor device 120 can provide an access event.

In certain example embodiments, the smart monitor device 120 records each access event, such as each open/close access event, in an accessible storage unit, such as the access event log 124 of the data storage unit 123. For example, the data monitoring module 121 of the smart monitor device 120 can include an entry in the access event log 124 that a cap including the smart monitor device 120 was opened (i.e., a first access event). The data monitoring module 121 of the smart monitor device 120 can also separately record an entry in the event log 124 that the cap in including the smart monitor device 120 was closed (i.e., a second access event). In other example embodiments, the smart monitor 120 may not distinguish between open and close points. In such example embodiments, the data monitoring module 121 may only determine that the container including the smart monitor device 120 was accessed 1, 2, 3, 4, 5 or more times, for example, without determining whether each access was an open event or close event.

In block 610, the smart monitor device 120 associates one or more timestamps with each access event. That is, for each access event, the data monitoring module 121 of the smart monitor device 120 records the time associated with the access event, such as via a clock associated with the smart monitoring device. For example, if the smart monitor device 120 is included as part of a medication cap and the data monitoring module 121 determines an open event as described herein for the cap, the data monitoring module 121 determines the time of the open access event. The data monitoring module 121 then associates the time with the access event, such as in the event log 124 of the smart monitor device 120.

Likewise, when the data monitoring module 121 determines a close access event as described herein, the data monitoring module 121 can determine the time of the close event and associate the time with the close event in the event log 124. For example, if the example medication cap is opened at 8:53:49 and closed at 8:54:02, the open access event can be recorded as 8:54:49 with the close access event be recorded as 8:54:02. Hence, in such example embodiments the access event includes the a first (open) time and a second (closed) time that corresponding to the opening and closing a cap, for example, that includes the smart monitor device 120. In example embodiments where the smart monitor device 120 does not distinguish between open/close events, for example, the smart monitor device 120 can associate the time that smart monitor device 120 detected each access event associated with the smart monitor device 120, i.e., the open event time and the close event time.

In certain example embodiments, the data monitoring module 121 of the smart monitor device 120 can also determine timestamped events such as when the smart monitor device 120 connects or disconnects to the user device 110. The data monitoring module 121 may also record the duration of connected or disconnected time. Likewise, the data monitoring module 121 of the smart monitor device 120 can, in certain example embodiments, determine the time that a refill request was received, such was when the user clicked or double clicked a button or other input associated with the smart monitor device 120. For example, the time of the click can be recorded, such as in the data storage unit 120 of the smart monitor device 120.

In block 615, the smart monitor device 120 communicates the dose adherence data to the dose management system 130 via the user device 110. That is, after determining the access events and associated times for each access event, the data monitoring module 121 communicates the time-stamped access events to the dose management system 130, the time-stamped access events corresponding to the adherence data. For example, the data monitoring module 121 of the data processing module 131 can communicate the access event log 124 entries and associated timestamps to the user device 110 as described herein via the short-range data connection. The user device 110 can then communicate the access event log 124 entries and associated time stamps to the dose management system 130 via the network 105. The dose management system 130 then receives the access event log 124 entries and associated timestamps. In certain example embodiments, the communication can occur via a low power cellular connection (e.g., NB-IoT).

In certain example embodiments, the smart monitor device 120 can be configured to communicate the adherence data at predetermined times, such as specific times during the day. Additionally or alternatively, the smart monitor device 120 can be configured to communicate the adherence data each time the smart monitor device 120 re-connects with the user device 110. For example, if the user 101 has moved the smart monitor device 120 out of range of the user device 110 such that a short-range connection cannot occur, when the user 101 moves the smart monitor device 120 back in to range—and the smart monitor device 120 re-connects to the user device 110—the smart monitor device 120 can communicate the adherence data to the user device 110. The user device 110 then receives the adherence data and communicates the adherence data to the dose management system 130 as described herein. Additionally or alternatively, the data processing module 131 can be configured to communicate adherence data to the dose management system 130 at or near the time of a scheduled dose, such as at one or more times after the scheduled dose.

Additionally or alternatively, the data monitoring module 121 of the smart monitor device 120 can be configured to communicate each time-stamped access event to the dose management system 130 on a rolling basis. For example, each time the data monitoring module 121 determines an access event the data monitoring module 121 can communicate the access event and associated timestamp (i.e., adherence data) to the user device 110, which then communicates the access event and associated timestamp to the dose management system 130 as described herein. The dose management system 130 then receives the adherence data. In certain example embodiments, the data monitoring module 121 can also communicate other information to the user device 110 as described herein, such as time-stamped connect/disconnect time and refill request times. The user device 110 can then communicate such information to the dose management system 130 via the network 105 and the dose management system 130 receives the information.

In block 620, the smart monitor device 120 optionally clears the adherence data memory. That is, after the smart monitor 120 communicates the adherence data to the user device 110, the data monitoring module 121 optionally erases any stored access events. The data monitoring module 121 may also erase any other stored, time-stamped information. For example, the data monitoring module 121 erases the access event log 124, thereby freeing storage space for event data. In certain example embodiments, the data monitoring module 121 erases the adherence data daily, such as after all adherence data has been communicated to the dose management system 130. Additionally or alternatively, the dose management system 130 can be configured to erase the adherence data as needed to maintain available storage space, such as when storage space becomes about 50%, 55%, 60%, 70%, 75%, 80%, 85%, 90%, or 95% full. In certain example embodiments, the data monitoring module 121 erases the access event log 124 only in response to receiving instructions to do so from the dose management application 112 on the user device 110 and/or from the dose management system 130.

Returning to FIG. 5, in block 515, the dose management system 130 compares the adherence data to the one or more management parameters to identify a possible missed dose. That is, the data processing module 131 of the dose management system 130 can read the content of the received adherence data. Then, by comparing the content of the received adherence data to the scheduled doses provided in the management parameters, the data processing module 131 can determine whether a dose may have been missed (or not). For example, if the management parameters provide that a dose of a medication is to be taken at 9:00 a.m., the data processing module 131 can, by reading the received adherence data, determine if an access event occurred at around 9:00 a.m. If so, the data processing module 131 can determine that a dose was likely not missed, as the access event at or near the scheduled dose provides a determined adherence indication for the scheduled dose (i.e., an indication that the user 101 likely took the scheduled dose). For example, if a dose was scheduled to be taken at 9:00 a.m., and the data processing module 131 identifies an open access event at 8:55, then the data processing module 131 can determine that a dose was likely taken around the scheduled time of 9:00 a.m. In other words, the identified open access event at 8:55 provides an adherence indication that the dose was taken.

Likewise, if pool chemicals are to be added to a pool each morning at 10:00 a.m., and the data processing module 131 identifies an open access event from the adherence data at 10:03 and a close access event at 10:04, the data processing module 131 can determine that a chemical dose to the pool was likely not missed. For example, both the identified open access and the close access event provide an adherence indication that the chemicals were administered. Alternatively, the absence of an adherence indication at or near the time of the scheduled dose indicates that a dose might have been missed, thus prompting the data processing module 131 to conduct further analysis as described herein.

In certain example embodiments, such as when smart monitor device 120 may be integrated as part of a cap and where the smart monitor device 120 can distinguish between open/close events, a combination of an open access event and a close access event at or near a scheduled dose time may provide a stronger adherence indication than a single open or close access event. For example, such a combination, where identified, may suggest that the user both opened and then closed the cap. Such a combination thus provides two adherence indications—close in time to each other—thereby providing a strong suggestion that the user 101 took or administered a scheduled dose. For example, an open access event at 8:58:46 a.m. and a close access event at 8:59:06 a.m for a 9:00 a.m. scheduled dose can provide a strong indication that the user 101 took or administered the dose according to determined schedule.

In block 520, if the dose management system 130 determines, based on the received adherence data, that the user 101 likely did not miss a scheduled dose, then the method follows the “NO” branch of block 520 to block 525 of FIG. 5. That is, when the data processing module 131 identifies an adherence indication, then the method proceeds to block 525. But if the dose management system 130 determines that the user 101 may have missed dose based on the absence of an adherence indication, then the method follows the “YES” branch of block 520 to block 530 of FIG. 5, in accordance with certain example embodiments.

In block 525, when the dose management system 130 determines that a dose was likely not missed, then the dose management system 130 continues to follow the determined reminder schedule. For example, the data processing module 131 may not send any additional reminders that are not already scheduled, as the adherence indication for the scheduled dose suggests that the dose was timely taken. Hence, with the identification of the adherence indication, the dose management system 130 can continue to receive and monitor adherence data for the user 101, thereby determining if a future dose was likely missed.

In block 530, when the dose management system 120 determines a scheduled dose may have been missed, the dose management system 130 confirms that a scheduled dose was likely missed. For example, the data processing module 131 of the dose management system 130 confirms that the possible missed dose is not the result of a connection error over the network 105 or between the user device 110 and the smart monitor device 120. The data processing module 131 also determines whether an adherence indication exists within a dosage time interval. The details of block 530 are provided in further detail below in FIG. 7.

Turning to FIG. 7, provided is a block flow diagram depicting a method 530 for confirming that a dose of a product was likely missed, in accordance with certain example embodiments.

With reference to FIGS. 1 and 5-7, in block 705 of FIG. 7, the dose management system 130 requests connection data from the user device 110. That is, the data processing module 131 of the dose management system 130 sends a communication to the user device 110 over the network 105. The communication, for example, can instruct the user device 110 to provide any data or information regarding the connection status between the user device 110 and the smart monitor device 120. For example, the connection data can include any data that the user device 110 routinely records or that the user device 110 is configured to record, such as the time when the smart device 120 connects to the user device 110, the duration of the connection, and the end time for the connection. The data can also identify the type of connection, such as whether the connection was an RFID connection, Bluetooth connection or local Wi-Fi, for example. In certain example embodiments, the dose management application 112 of the user device 110 can be configured to obtain and record such information, such as in the data storage unit 113 of the user device 110. Based on the received request for the connection data, the dose management application 112, for example, can communicate the connection data to the dose management system 130 via the network 105. The dose management system 130 can then receive the connection data via the network 105.

In block 710, if a response to the request for connection data is not received, then the method follows the “NO” branch of block 710 to block 715. But if the user device 110 provides a response, i.e., the user device 110 responds to the request such as by providing the requested connection data, then the method follows the “YES” branch of block 710 to block 720, in accordance with certain example embodiments.

In block 715, if the dose management system 130 does not receive a response from the user device 110, the dose management system 130 attempts to notify user of the error and/or records the error. That is, if the data processing module 131 does not receive a response to the request for the connection data, the data processing module 131 can determine that a connection error exists with the connection between the dose management system 130 and the user device 110. Hence, the data processing module 131 can determine that the absence of an adherence indication was likely due to a connection issue (and not necessarily a likely missed dose). For example, perhaps the user 101 has logged out of the dose management application 112, and hence the dose management application 112 is not communicated connection to the dose management system 130. Or, perhaps the user 101 has shut the user device 110 down, such as while charging the device. Additionally or alternatively, the user 101 may have turned off the short-range connection means of the user device 110. For example, the user 101 may have turned off their Bluetooth connection. Additionally or alternatively, the user 101 may have moved the user device 110 out of connection range for an extended period of time.

If the connection data is not received, however, the dose management system 130 can, in certain example embodiments, attempt to contact the user 101 via other methods, such as via text message, email, and/or a telephone call. For example, the dose management system 130 (or an operator thereof) can communicate to the user 101 that there is a connection issue between the user device 110 and the dose management system 130, such that the user 101 can attempt to correct the issue. The user 101, for example, may need to log back in to the dose management application 112. In certain example embodiments, the dose management system 130 can take additional steps to determine whether the user 101 took or administered the product at the scheduled time, such as by asking the user 101 via the text message, email, and/or a telephone call. In certain example embodiments, the data processing module 131 can record the connection error, such as in a record of the data storage unit 134.

In block 720, if the dose management system 130 receives a response to the connection data request, the dose management system 130 verifies a smart monitor device 120 connection within a dosage time interval. That is, if the dose management system 130 receives a response from the user device 110, then the data processing module 131 of the dose management system 130 can determine that there is no connection issue between the dose management system 130 and the user device 110 (as the received response demonstrates successful connection of the dose management system 130 to the user device). Thereafter, by reading the received connection data, in certain example embodiments the data processing module 131 can analyze the connections between the user device 110 and the smart monitor device 120 to verify whether suitable connections between the user device 110 and the smart monitor device 110 have occurred. For example, the data processing module 131 can read the received connection data to determine if, when, and/or for how long the smart monitor device 120 has been connected to the user device 110. If the smart monitor device 120 and the user device 110 have not been connected for 24 hours, for example, then the data processing module 131 can identify a lack of connection as a connection error (rather than a likely missed dose).

Additionally or alternatively, if the data processing module 131 determines that the connection between the user device 110 and the smart monitor device 110 is sporadic and/or interrupted, the data processing module 131 can determine that the user 101 may be frequently moving the smart monitor device 120 in-and-out of range of the user device 110, thereby disrupting the connection and causing the connection error. And, based on identifying the connection error between the user device 110 and the smart monitor device 120, the data processing module 131 can determine that the connection error is more likely the cause of the missing adherence indication for a scheduled dose (rather than an actual missed dose).

Additionally or alternatively, the data processing module 131 of the dose management system 130 can determine if the smart monitor device 120 was connected to the user device 110 during a dosage time interval, thereby further determining the likelihood of a missed dose verses a connection error associated with the operating environment 100. The dosage time interval, for example, can be determined as described in block 215 of FIG. 2. For example, if the product is a prescription medication that requires a dose be taken at 9:00 a.m. and 6:00 p.m. each day, then the data processing module 131 may determine, as part of the management parameters discussed above, that the dosage time intervals are 7:00 a.m. to 11:00 a.m. for the 9:00 a.m. dose. Hence, if the data processing module 131 thereafter determines in block 720 of FIG. 7 that one or more connections occurred between the user device 110 and the smart monitor device 120 during the dosage time interval—such as at 9:38 a.m.—then the data processing module 131 can determine that the connection between the user device 110 and the smart monitor device 120 was active and working during the dosage time interval. And, based on the successful connection of the user device 110 and the smart monitor device 120 during the dosage time interval, the data processing module 131 can also determine that the missing adherence indication was less likely the result of a connection error (i.e., more likely a missed dose).

In block 725, if the data processing module 131 of the dose management system 130 determines that a connection error exists between the smart monitor device 120 and the user device 110—and/or that the smart monitor device 120 and the user device 110 were not connected during a dosage time interval—then the method follows the “NO” branch of block 725 back to block 715. That is, if a connection error exists between the smart monitor 120 and the user device 110, then the data processing module 131 of the dose management system 130 can attempt to contact the user 101 so that the user 101 can take steps to correct the connection error. For example, the user 101 may need to bring the smart device monitor 120 back into range of the user device 110 so that the short-range connection can be re-established, thereby permitting the communication of the adherence data as described in block 510 of FIG. 5. The data processing module 131 can also attempt to contact the user 101, for example, via text message, email, and/or a telephone call as described herein, to inform the user 101 of the connection errors.

Alternatively, if the data processing module 131 of the dose management system 130 determines—by reading the received connection data—that there is no connection error between the user device 110 and the smart monitor 120, then the method follows the “YES” branch of block 725 to block 730. That is, in the absence of an adherence indication as described herein and in the absence of a connection error between the between the user device 110 and the smart monitor 120, the data processing module 131 can determine that a connection error is less likely to be the cause of the missing adherence indication (and that hence the cause is more likely a missed dose).

In block 730, the dose management system 130 determines whether an adherence indication is present within a dosage time interval. That is, the data processing module 131 of the dose management system 130 can expand the window in which the data processing module 131 determines an access event, thereby possibly identifying an adherence indication further in time before or after the scheduled dose. If a dose was scheduled at 9:00 a.m., for example, and the data processing module 131 does not identify an adherence indication between 8:55 a.m. to 9:05 a.m., the data processing module 131 can—based on reading the adherence data—determine whether an access event occurred at a time before or after the 8:55 a.m. to 9:05 a.m. period. For example, the data processing module 131 can determine if an adherence indication is present during a dosage time interval from 8:00 a.m. to 10:00 a.m. For the scheduled 9:00 a.m. dose, for example, the data processing module 131 may identify an access event at 9:53, thus identifying an access event (and hence an adherence indication) within the dosage time interval.

By expanding the period in which the data processing module 131 of the dose management system 130 attempts to identify an adherence indication to a dosage time interval, the data processing module 131 can, in certain example embodiments, better determine whether a possible missed dose was actually missed (or not). For example, if an adherence indication is not initially identified close in time to the scheduled dose, failing to expand the search window in which the dose might have been taken or administered may result in incorrectly identifying the dose as a likely missed dose. Expanding the search window, however—by configuring the data processing module 131 to search for an adherence indication within a predetermined dosage time interval, for example—can reduce the likelihood of incorrectly identifying a possible missed dose as an actual missed dose. Rather, the possible missed dose may have been taken earlier or later than scheduled, in which case the presence of an adherence indication within the dosage window can identify the possible missed dose as a dose that was likely taken.

In block 735, if the dose management system 130 identifies an adherence indication within a dosage time interval, then the method follows the “YES” branch of block 735 to block 740. But if the if the dose management system 130 does not identify an adherence indication in the dosage time interval, the methods follows the “NO” branch of block 735, returning to block 535 of FIG. 5.

In block 740, if the data processing module 131 of the dose management system 130 determines that an adherence indication is present within the dosage time interval, in certain example embodiments the data processing module 131 ceases the analysis for the possible missed dose. That is, based on identifying an adherence indication within the dosage time interval, the data processing module 131 determines that the dose was likely not missed and hence that a reminder for the missed dose does not need to be sent to the user 101. In other words, while the data processing module 131 initially can identify a dose as a possible missed dose (based on a missing adherence indication), such as in blocks 515 and 520 of FIG. 5, the further analysis of the adherence data can identify an adherence indication during the dosage time interval, thereby indicating that the dose was not actually missed.

Returning to FIG. 5, in block 535, if the dose management system 130 does not identify an adherence indication within the dosage time interval, the dose management system 130 the user 101 of the likely missed dose. That is, if the data processing module 131 of the dose management system 130 does not locate an adherence indication, either initially in (as in blocks 515 and 520) and after further analysis as in block 730, then the dose management system 130 determines that the scheduled dose was very likely missed. Hence, the data processing module 131 communicates a “missed dose” alert to the user 101.

In certain example embodiments, the data processing module 131 can send a text message to the user device 110 via the network 105, the text message notifying the user 101 of the likely missed dose. Additionally or alternatively, the data processing module 131 may trigger a telephone call, such as an automated call, to the user device 110, the call informing the user 101 of the likely missed dose. Additionally or alternatively, the data processing module 131 may communicate an instruction to the smart monitor device 120 via the user device 110 and the network 105, the instruction causing the smart monitor device 120 to provide an audio and/or visual alert to the user 101 regarding the missed dose. For example, the instruction may case the smart monitor device 120 to beep, vibrate, and/or providing a blinking light, etc., thereby notifying the user 101 of the missed dose. In certain example embodiments, the dose management system 130 may can also communicate the “missed dose” the user's healthcare provider, thereby permitting the healthcare provider to intervene with the user's adherence to the dose schedule for the product.

In certain example embodiments, the dosage time interval described herein can be narrowly configured to determine whether a dose was taken or administered precisely as scheduled. For example, if a medication needs to be taken as close to 9:00 a.m. each day as possible, the dosage time interval can be set narrowly at 8:55 to 9:05. If an adherence indication is identified at 9:02 a.m., for example, then the dose management system 130 can determine—with fairly high confidence—that the dose was taken very close to the scheduled 9:00 a.m. time (i.e, within two minutes of the scheduled time). But if an adherence indication is determined as described herein at 9:23 a.m., the dose management system 130 can determine that the dose was likely taken or administered, but that the dose was taken or administered late. The dose management system 130 can then communicate the late dose information to the user 101 and/or the user's healthcare provider, for example, thereby permitting the user 101 and/or the user's healthcare provider to address the untimely dose. In other example embodiments, if no adherence indication is received for the 9:00 a.m. dose—and the data processing module 131 verifies connections between the user device 110 and the smart monitor device 120 at 9:05 a.m., 9:16 a.m., and 9:30 a.m.—then the data processing module 131 can determine with relatively high confidence that the missing adherence indication was not do to a connection error but rather a missed dose. The data processing module 131 can then remind the user 101 to take the dose as described herein and/or notify the user's healthcare provider, for example, of the missed dose.

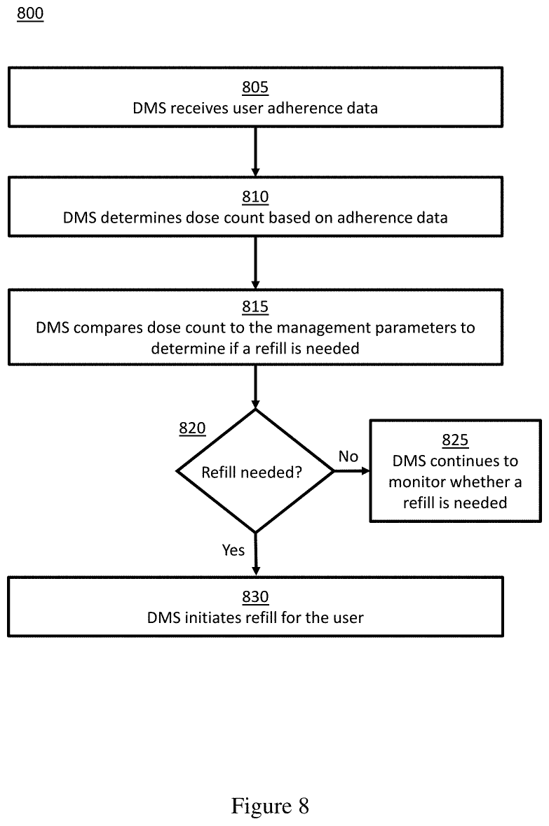

FIG. 8 is a block flow diagram depicting a method 800 for providing a refill based on a determined dose count, in accordance with certain example embodiments.

With reference to FIGS. 1, 4-6, in block 805 of FIG. 8, the dose management system 130 receives user adherence data. That is, the dose management system 130 receives the user adherence data according to block 510 of FIG. 5 and FIG. 6. For example, the smart monitor device 120 determines access events associated with the smart monitor device 120. The access events can include, for example, open/close events where the user 101 has opened a container including the smart monitor device 120 and then replaced the smart monitor device 120 on the container, as described herein. The smart monitor device 120 then communicates the access event information to the user device 110 via a short-range connection, and the user device 110 then communicates the access event information to the dose management system 130 via the network 105. The dose management system 130 then receives the access event information as the adherence data, via the network 105.

In block 810, the dose management system 130 determines a dose count based on the adherence data. That is, the data processing module 131 reads the received adherence data to determine an estimate for the number of doses that have been taken or administered from the container including the smart monitor device 120 relative to a dose start time. For example, the data processing module 131 can determine an estimate of the number of doses that have been taken or administered from the container including the smart monitor device 120 since the filled container was provided to the user 101, since the smart monitor device first provided an adherence indication, or since the last refill was provided to the user 101.

To determine the dose count, for example, the data processing module 131 reads the adherence data to identify the number of adherence indications relative to a dose start time. For example, because each adherence indication can provide an indication that the user 101 was accessing the container including the smart monitor device 120—and presumably taking or administering a dose from the container—the adherence indication can provide an indication of a taken or administered dose as described herein. If the data processing module 131 identifies 60 adherence indications on day 30 since a refill was first provided to a user 101, then the data processing module 131 can determine a dose count of 60 doses over the 30-day period. Hence, the number of doses (60 in this example) is determined relative to the dose start time 30 days earlier. Likewise, if the data processing module 131 identifies 90 adherence indications on day 30 since a refill was first provided to a user 101, then the data processing module 131 can determine a dose count of 90 doses over the 30-day period.

In certain example embodiments, the data processing module 131 can analyze the adherence data to determine open/close access events that are close in time to each other, thereby better estimating the dose count. For example, for a given dose of a medication, a user 101 may open a cap including the smart monitor device 120, retrieve the medication, and close the cap. If the data processing module 131 identifies both the open access event and the close event as separate adherence indications that are close in time, the data processing module 131 can treat the adherence indications as a single data point for the dose count determination. For example, for a given dose, such as a dose scheduled at 9:00 a.m., the data processing module 131 may identify an open access event at 9:02:23 a.m. and a close event at 9:03:18. Rather than treating each access evet as a separate adherence indication (and hence each a taken or administered dose), the data processing module 131 could be configured to count the open/close events as a single dose for the dose count. For example, the open access event at 9:02:23 a.m. and a close event at 9:03:18 would be counted as a single dose for the dose count. In certain example embodiments, the data processing module 131 may determine a dose count over a period of time, such as over 30 days or a month, without regard to whether the user 101 first accessed a container including the smart monitor device 120 on day 1 or when the user 101 first received the product.

As those skilled in that will appreciate based on this disclosure, counting two access events close in time to each other as a single dose event can, in certain example embodiments, prevent over-counting of likely doses by the data processing module 131. For example, if a medication is to be taken every day at 9:00 a.m., with 30 doses initially provided to the user 101 and 12 refills available, counting each open/close access event as a single dose would, after 30 days, indicate that 30 doses have been taken. But if each open access event and each close access event were counted separately as a dose, 30 doses would incorrectly be identified as being taken or administered by day 15. That is, the data processing module 131 may incorrectly count 30 doses taken over 15 days, when in fact the user 101 had taken 15 doses over 15 days. Hence, counting each open/close access event as a single dose can, in certain example embodiments, increase the accuracy the estimated dose count.

In certain example embodiments, the data processing module 131 of the dose management system 130 can subtract one or missed doses from the dose count. For example, if the dose management system 130 identifies a possible missed dose, such as described according to the methods of FIGS. 5-7, the data processing module 131 can deduct the missed dose from the dose count. If the dose is a pool chemical that needs to be added to a pool each day, and the data processing module 131 identifies 3 missed doses over the course of 30 days, then the data processing module 131 can determine the dose count as 27 doses on day 30, thereby accounting for the missed doses.

In block 815, the dose management system 130 compares the dose count to the management parameters to determine if a refill is needed. That is, using the determined dose count, the data processing module 131 of the dose management system 130 can determine whether a refill request should be initiated, such as based on whether the amount of the product being managed is running low. To determine whether a refill is needed, the data processing module 131 can read the one or more management parameters to determine the number of doses the user 101 is to take or administer over a given amount of time, such as 30 doses per month. For example, if the management parameters provide that a medication is to be taken or administered every day at 9:00 a.m. for 30 days, with 12 refills total, the data processing module 131 can read the management parameters as described herein to identify such dosage information. Thereafter, such as at day 25 after the first filled prescription is provided to the user 101 in a container including the smart monitor device 120, the data processing module 131 may determine a dose count of 25, thereby indicating that the user 101 is taking the medication each day as prescribed. And, by comparing the dose count of 25 doses to the 30 doses initially provided to the user 101, the data processing module 131 can determine, for example, that the user 101 should only have 5 doses remaining before the initial 30 doses are used up. Hence, the data processing module 131 can determine that a refill should be initiated on behalf of the user 101 based on reading the adherence data, determining the dose count, and comparing the dose count to the management parameters for the product. In certain example embodiments, the dose management system 130 can also determine whether the user 101 is eligible for the refill, such as described in block 415 and 420 of FIG. 4.

In block 820 of FIG. 8, if the dose management system 130 determines that the user is not in need of a refill, then the method follows the “NO” branch of block 820 to block 825. But if the dose management system 130 determines that a user 101 should receive a refill, such as when the product that is the subject of the dose regimen is running low and/or is about to run out, then the method follows the “YES” branch of block 820 to block 830.

In block 825, based on determining that the user 101 does not yet need a refill, then the dose management system 130 continues to monitor whether a refill is needed. That is, the data processing module 131 continues to determine the dose count from the adherence data as described herein and compare the determined dose count to the management parameters. For example, if the one or more management parameters require 30 doses over 30 days, and the data processing module 131 determines on day 15 after the product is provided to the user 101 that 15 doses have been taken, then the data processing module 131 can determine that a refill is not yet needed (as there should be 15 doses remaining). Thereafter, however, the data processing module 131 can continue to determine the dose count and compare the dose count to the number of doses per unit time (e.g., doses per month) identified in the management parameters as described herein until the data processing module 131 determines that a refill is needed.

In block 830, when the dose management system 130 determines that a refill is needed, the dose management system 130 initiates a refill for the user 101. That is, the data processing module 131 of the dose management system 130 requests a refill for the user 101, such as described in block 430 of FIG. 4 described above. For example, the dose management system 130 notifies an operator of the dose management system 130 and/or the provider system 140 that the product being managed needs to be refilled. The operator can then fulfill the request, thereby processing the refill for the user 101. In certain example embodiments, the dose management system 130 can also receive an input confirming that the refill request has been processed, update the management parameters to identify the refill, and notify the user that the refill request has been fulfilled, such as described in blocks 435-445 in FIG. 4.

In certain example embodiments, the dose management system 130 can use the dose count to also monitor proper product usage, i.e., that the user 101 is adhering to the management parameters. For example, the management parameters may provide that a user 101 is to take or administer a dose of a medication each day for 30 days. If a pharmacist provides the user 101 with 30 initial doses, but on day 15 since the user 101 first received the medication the data processing module 131 determines that 18 doses have likely been taken, the data processing module 131 can determine that the user 101 may be taking more medication than prescribed. For example, on day 15 the user 101 would be expected to have taken 15 doses, not 18. Hence, the data processing module 131 of the dose management system 130 can notify the user 101 and/or the user's healthcare provider regarding the possible overuse of the medication.

Likewise, in certain example embodiments, the data processing module 131 can determine from the adherence data and the dose count that the user 101 may be taking or administering too few doses. For example, if on day 15 in the example above the user 101 has only taken 9 doses based on the analysis of the adherence data, then—in addition to sending reminders—for example, the data processing module 131 can determine that user 101 is likely not adhering to the management parameters. The dose management system 130 can then notify the user 101 and/or the user's healthcare provider, for example, of the lack of adherence as described herein.

In certain example embodiments, the dose management system 130 may only monitor the doses of a product being used and then send a refill. For example, if the product is a laundry detergent, the management parameters may indicate that that 1 cup of detergent should be used for each load of laundry, and that 30 cups are provided in the laundry detergent container (the container lid including a smart monitor device 120). Hence, after the data processing module 131 determines from the methods and systems described herein that 25 doses of the detergent have likely been used, the data processing module 131 can process a refill request for the user. In such example embodiments, the data processing module 131 can determine that a refill is needed, whether the doses of the detergent have been administered over 10 days or 45 days. In other words, the relevant indicator that a refill needs to be sent is that the product is running out (e.g., 25 of 30 doses likely used).

In certain example embodiments, the connections and communications between the smart monitor device 120 and the user device 110 described herein can be configured to conserve power on the smart monitor device 120. That is, the smart monitoring device 120 can be configured for a low-power management, thereby providing improved battery life and performance within a small footprint. For example, the smart monitoring device 120 may emit wireless data transmission packets at a first, longer and slower interval a majority of the time, but it may emit wireless data transmission packets at a second, shorter and faster interval in response to either a) the predicted dose schedule or b) data collected by the sensor on the smart monitoring device 120. In certain example embodiments, the first, slower interval between wireless data packet transmissions may be between 200 and 2000 ms, and the second, faster interval between wireless data packet transmissions may be between 20 and 500 ms.

In the example of a smart medication cap including the smart monitoring device 120, the faster advertisement may occur for a period of 5-500 seconds after opening of the cap, or after a scheduled dose/alarm. The energy being sent to the wireless radio antenna may be increased during this period in order to increase the transmission range. This can be beneficial in terms of improving the probability of establishing a connection with a smart monitoring device that may be on the edge of a transmission range, such as may occur when a smart monitoring device 120 is located on one side of a user's house, with their user device 110 located on the opposite side of the user's house. Fast advertisement periods or increased transmission energy periods may also be cycled through periodically as a way to improve the probability of connection.

In certain example embodiments, when a wireless advertisement packet is received and recognized by a user device 110, a connection sequence is established between the smart monitor device 120 and the user device 110. This connection may be dependent, at least in part, on the token that is communicated by the smart monitor device 110. As part of this process, the smart monitor device 120 may request agreed-upon connection parameters to be established with the user device 110. For example, the smart monitor device 120 may request a specific min/max connection within an agreed upon range, such as 65-85 ms, a child-device latency, and a connection supervisor timeout. These connection parameters may be used in combination with real-time clocks embedded on the smart monitor device 120 and the user device 110 in order to permit distributed communication on a local, low-power network, such as a network that may be run at a 2.4 GHz frequency.

In certain example embodiments, in order to keep the connection open and as an additional security feature, after a connection is formed between the user device 110 and the smart monitor device 120, a “Keep-Alive” key or token can be sent from the user device 110 to the smart monitor device 120. If this “Keep-Alive” key is not received within a pre-defined period of time, such as within about 5, 10, 15, 20, 30, 40, 50, or 60 seconds, then the smart monitor device 120 may drop the connection with the user device 110. For example, the dose management system 130 requests, via the network 105 and the user device 110, to connect to the smart monitor device 120. If the connection is successful, then the dose management system 130 communicates the keep alive key to the user device 110, which then communicates the key to the smart monitor device 120. The purpose of the keep alive key is to confirm that the connection to the smart monitor device 120 is from an authorized remote computer. If the keep alive key is received within a specified period of time, then the smart monitor device 120 stays connected. If the keep alive key is not received, then the smart monitor device 120 is disconnected and optionally goes into hibernation.

In certain example embodiments, the interval between wireless data packet transmissions may be changed after a connection is established between the user device 110 and the smart monitor device 120. In addition, the transmit power of a low-power, short range radio may also be altered after a connection is established. When a connection drops, such as may occur when a smart monitor device 120 is moved out of wireless transmission range of the user device 110, then the smart monitor device 120 may reduce or increase its transmit power.

When a connection is open the smart monitor device 120 may periodically send default log “ping” events on a predefined interval to the user device 110 as a means of sending an update on device status. As an example, these events may include the device token, a timestamp, the connection status, the battery life, or any other information that may relate to the overall status of the connection. The user device 110 may store such ping events in memory or relay data sent in such ping events to the dose management system 130. Ping events may be sent on a predefined interval, such as every 10-20 minutes, or they may be sent on a semi-randomized time periods that are defined by an algorithm that is programmed into the embedded software on the smart monitor device 120.

The smart monitor device 110 may store a characteristic in memory on data storage unit 123 that signifies whether all data stored on the smart monitor device 120 has been successfully synced to the user device 110. When a connection is open, the log of event data may be wirelessly sent by the smart monitor device 120 to the dose management application 112 running on the user device 110. When this data is successfully received and stored in the data storage unit 113 on the user device 110, the user device may wirelessly send a characteristic to the smart monitor device 120 indicating that data has been successfully stored. When the smart monitor device receives this characteristic it may mark data that is stored in the event log as “synced”. Optionally, the embedded software running on the smart monitor device 120 may delete any data marked as synced from the data storage unit 123 on the smart monitoring device 123. Optionally, as a way to conserve power the smart monitor device 120 may also drop the connection with the user device 110 and reduce wireless data transmission packet advertising frequency and/or reduce wireless data transmission energy when the event log is clear or when all data in the event log is marked as “synced”.

In certain example embodiments, an increase or reduction of wireless data transmission energy may be achieved by altering the voltage or current permitted to be sent to the wireless radio module that is in electrical communication with the circuit board on the smart monitor device 120. This capability can be governed, at least in part, by embedded software running on the microprocessor that is incorporated into the smart monitor device 120.

### Example Smart Monitor Device

### Outer Housing

In certain example embodiments, provided is an example smart monitor device 120 that can be used and relied on, for example, in accordance with the methods and systems described herein.

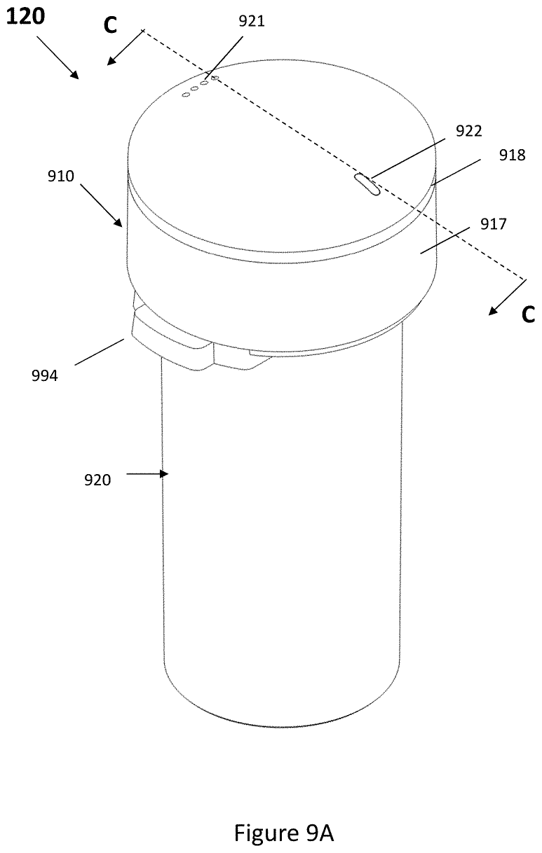

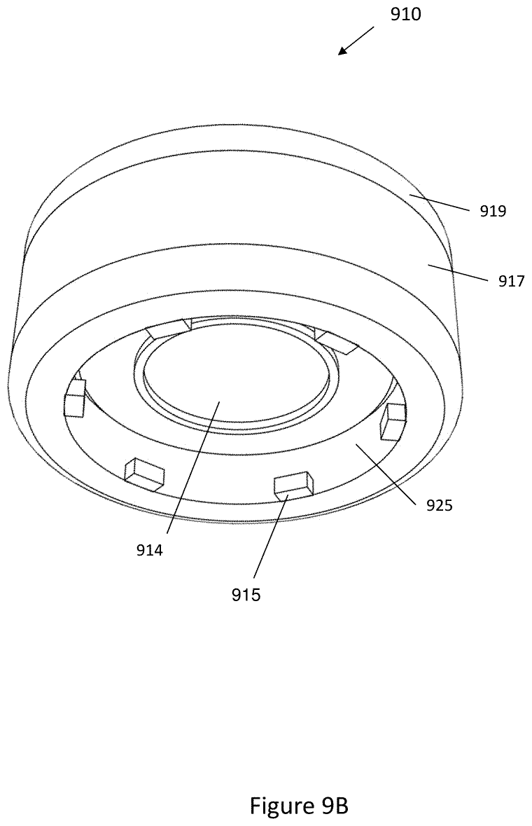

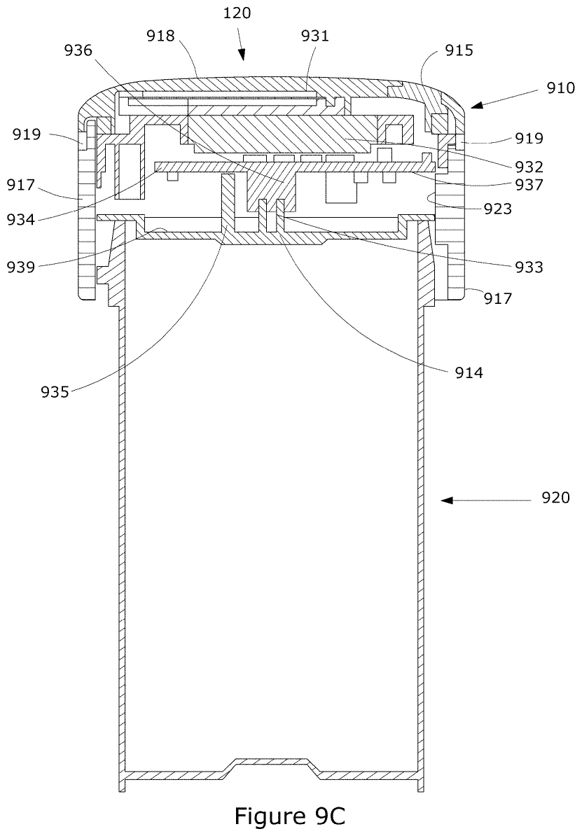

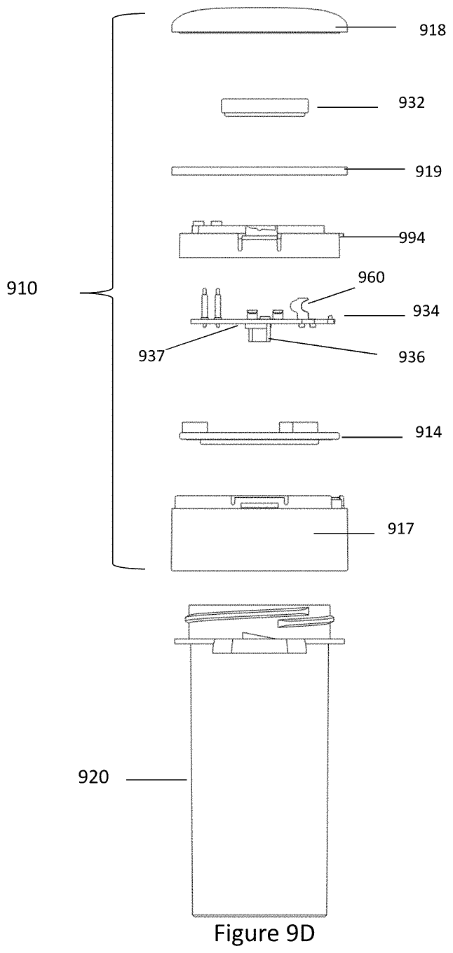

With reference to FIGS. 9A-11G, the example smart monitor device 120 comprises a cap or lid unit 910, that is able to be removably connected to a container unit 920 that stores tangible items (e.g. consumable user products, such as pills), requiring a networked computer system as disclosed herein to automatedly monitor access to the items. The cap or lid unit 910 comprises the hardware, software, and electrical components used in the methods disclosed herein, e.g. for the smart monitor device 120 to wirelessly communicate with the user device 110 data comprising opening/closing access events of the cap unit 910.

In one embodiment, the smart monitor device 120 comprises the cap unit 910 without the container unit 920. Non-limiting examples of smart monitoring devices 120 comprising a cap or lid unit 910 without a container unit 920, comprise smart devices with a variety of shapes, that attach directly to, or communicate wirelessly with, another user device, e.g. a health monitoring device, such as a blood glucose meter, blood pressure cuff, an inhaler, a scale, a heart rate monitor, a blood alcohol reader, a hearing aid, smart eyeglasses, a smart watch, a thermometer, and the like.

In another embodiment, the smart monitor device comprises the cap unit 910 and the container unit 920. Various smart monitor device 120 shapes and uses are envisioned with the scope of the present invention. The shape of the device 120's outer housing, comprising the cap unit 910 and the container 920, is dictated at least in part by the size and shape and/or maximum volume of the items (e.g. pills) that are securely housed in the container unit 920. In the pill container embodiment of FIGS. 9A-11G, the device 120 is designed to be of a size where it is compatible with existing pharmacy robotic filling systems.

Non-limiting examples of container units 920 suitable for fitting a smart cap unit 910 to comprise: a container for pills or tablets, a container for vitamins or supplements, an inhaler, a syringe, a container for liquid medication, a container for pool or water treatment chemicals, a container for industrial chemicals, a container for lab chemicals, a container for food or food ingredients, a container for aircraft or machine maintenance fluids, a cosmetic cream or lotion a container for facewash or shampoo, a container for mouthwash or toothpaste, an eye drop container, a nasal spray container, pet food containers, a container for baby food, a cigarette container, trash receptacles, mailboxes, cleaning supplies packaging, a container for cannabis products, a container for candy or breath mints, infant formula container, nutritional food packaging, water bottles, a container for coins or money, a container for laundry detergent, beverage containers, a container for gasoline, a container for holding additional interior packaging or a container for any other consumable good.

An exemplary embodiment of the smart monitor device 120 comprises a smart cap with a sliding non-flexible plate, and a pill container, as disclosed in US Patent Application 20160048657 A1 to the present inventors. In another embodiment, as disclosed in FIGS. 9A-11G herein, the smart monitor device 120 comprises a cap unit 910 with a push-plate 914, which is a flexible plate that is linearly translatable within the cap, and a pill container 920, able to automatedly order a refill by detecting a user pushing and releasing the cap unit 910 twice, in rapid succession.

The outer housing of a cap unit 910 of FIGS. 9A-9D, comprises a top portion, such as a cover 918; and a bottom portion comprising an adapter ring 917 that holds the push-plate 914 in position. The bottom portion may further comprise an optional seam cover 919 to conceal the joint between the cover 918 and the adapter ring 917. The cover 918 is a thin circular disc that is removeable to expose the underlying battery 932, such as for battery replacement.

In an example embodiment, adapter ring 917 is a plastic ring with a selected internal thread pattern matching (i.e. configured to cooperate with) a container unit 920 external thread pattern. Adapter ring 917 can also be swapped out to fit multiple container unit 920 types (of similar diameter) without changing the other smart device components.

FIG. 9B is an illustration of an exemplary embodiment of a container-receiving cavity 925 defined by the cap unit 910. In FIG. 9B, the seam cover 919, adapter ring 917, a plurality of cap protrusions 915 protruding radially inward from a surface of the adapter ring 917 that defines the inner diameter of the adapter ring (and hence the outer diameter of the container receiving cavity 925), and a push-plate 914 defining a closed end of the container-receiving cavity 925 are visible. The push-plate 914 is positioned horizontally relative to the cover (not shown), and perpendicularly relative to the adapter ring 917.

FIG. 9C is a cross-sectional view of an exemplary smart monitor device 120 comprising the cap unit 910 on a pill container unit 920. Cover 918 includes an indicator or indicator window 915 for a visual indication to a user, such as a light emitter, and conceals a piezo buzzer 931 within an internal cavity 923 defined by the cap unit 910. The piezo buzzer 931 may lie directly beneath cover 918. The outer housing of the cap unit 910 further comprises the adapter ring 917 on the cap's bottom portion, with the seal 919 disguising the seam between ring 917 and cover 918. Battery 932 is housed within the internal cavity 923 to power the onboard circuitry, such as a PCB 934. The battery 932 may lie beneath the piezo buzzer and atop the PCB 934. A switch 936, which is in electrical communication with the PCB 934, extends from an underside 937 of the PCB 934 that faces the push-plate 914 and extends toward the push-plate 914. The switch 936 may be integral with or connected to the PCB 934, and in one embodiment may be a depressor switch.

The push-plate 914 has a switch activator 933 and at least one stop 935 extending from an upper-side 939 thereof, which faces the underside 937 of the PCB 934. Each stop 935 has a predetermined length that enables the switch 936 to be activated or the switch 936 to be depressed a safe distance without applying a pressure to the PCB 934 that could damage or break the board. Each stop 935 has a free end that moves into direct contact against the underside 937 of the PCB 934 as a linear translation limiter for the push-plate 914. The switch activator 933 has a shorter length relative to each stop and is positioned in alignment with the switch 936 to activate the same when the push-plate is pushed (moved) toward the PCB 934, which has a fixed position within the internal cavity 923 of the cap unit 910. In one embodiment, the switch activator 933 has a depth of linear translation of about 0.1 to 2.5 mm into the switch 936.

FIG. 9D is an exploded view of FIG. 9C showing the external device 120 housing components (918, 917, 920), and the internal cap unit 910 components. From top to bottom, the cap unit 910 comprises a cover 918 in the shape of a thin disc, covering a coin shaped battery 932, a seam cover 919, a thumb release mechanism 994 to assist in removing the cap unit, PCB 934 with a switch 936 attached on the underside, a flexible push-plate 914, and the adapter ring 917.

### Switch Detector Sensor and Push-Plate

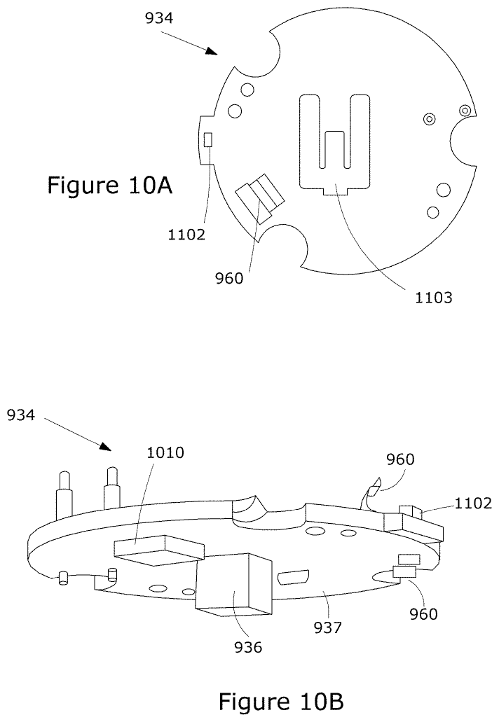

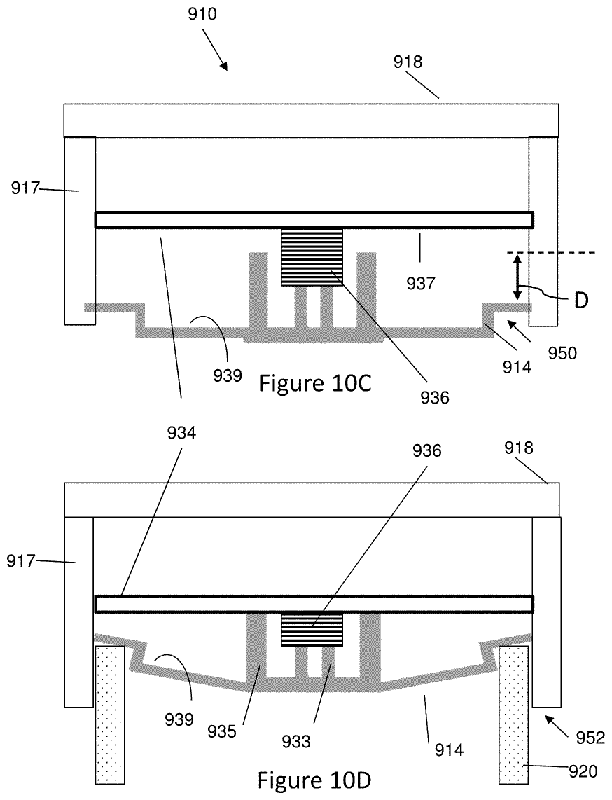

The smart monitor device 120 further comprises a contact sensor connected to, or in communication with, the PCB 934 to trigger an electrical signal to a microprocessor on the PCB 934 whenever the device 120 is opened (e.g. the cap unit 910 is removed from the container 920), and when the device is closed (e.g. the cap unit 910 is placed on container 920 and pressed down upon). As illustrated in FIG. 10B, the sensor comprises a depressor switch 936 connected to the bottom side of the PCB 934. The bottom end of the depressor switch 936 is touching a push-plate 914 in the assembled embodiment, as illustrated in FIGS. 10C and 10 D.

The push-plate 914 is received in the internal cavity 923 and closed the internal cavity opposite the cover and defines a container receiving cavity 925 within the interior of the adaptor ring 917. The push-plate 914 is linearly translatable within the internal cavity 923 a preselected distance D at a periphery of the plate and less than the preselected distance D at a center region of the plate, thereby elastically flexing the plate between the center region and the periphery. The push-plate 914 is flexible between 0 mm and 5 mm inward toward the cover 918 at the push-plate center when the cap is in a closed position of FIG. 10D, thus requiring the user to push down on the cap 910 to remove the cap from container 920. In the longitudinal cross-section of FIG. 10C, the plate has a stepped-shoulder with a flange extending radially outward therefrom to define a seat to receive a rim of a container mouth, as subsequently shown in FIG. 10D. The center region comprises a stop 935 and a switch activator 933 both extending from an upperside 939 of the plate facing the electronics, wherein the stop 935 has a predetermined length and the switch activator 933 has a length that is less than the predetermined length of the stop.

As illustrated in FIG. 10D, when the smart cap unit 910 is attached to the container unit 920, the depressor switch 936 is depressed by the flexible push-plate 914, triggering the attached microprocessor on the PCB 934 to read the device 110 as being in a “closed” position 952. When the cap unit 910 is removed from the container 920, the sensing switch 936 is released because the character of the push-plate in its flexed and/or elastically deformed condition inherently returns the push-plate back to its normal position 950 of FIG. 10C, triggering the attached microprocessor to read the vial as being “opened.”

In the various embodiments of the present invention, push-plate 914 extends the diameter of the internal cavity 923, and is between about 0.2 mm and about 2 mm thick, or between 0.5 mm and about 1.5 mm thick, or between about 0.1 mm to about 4 mm thick. The sliding plate is also about 1 mm thick in a preferred embodiment; and is made out of polypropylene or another food-grade polymer.

The push-plate 914 may also have a compressible seal on the ends that come in contact with the container 920 in order to create a tighter moisture-proof barrier that meets the requirements of moisture performance testing defined in USP 37 671.

### User Control Option for Refills

In an example embodiment, the smart monitor device 120 comprises a user control mechanism to automatedly order a smart monitor device refill when the user depresses and releases the cap unit 910 two sequential times, in rapid succession. The microprocessor is able to receive a signal from the switch 936 when compressed by the push-plate 914 upon a user depressing and releasing the cap unit. The microprocessor 1010 is configured to then store in memory information corresponding with the push-plate 914 pressing against the switch 936, the information indicative of the user “double tapping” the cap. The PCB radio then relays the information wirelessly to the user electronic computing device 110.

### Printed Circuit Board

Smart monitor device 120 further comprises a printed circuit board (PCB) 934 positioned within the internal cavity 923 of the cap unit 910 to enable the device 120 to detect access events and to trigger alarms while communicating wirelessly with the user electronic computing device (FIG. 1, 110). In the exemplified embodiment, the PCB 934 is positioned horizontally in the cap unit 910, extending the diameter of the cavity, beneath the cover 918 and above the push-plate 914. In an embodiment, PCB 934 is oriented perpendicular to an inner side wall of the cap unit 910, or horizontally relative to the cover 918. PCB 934 is between about 25 and 45 mm in diameter.

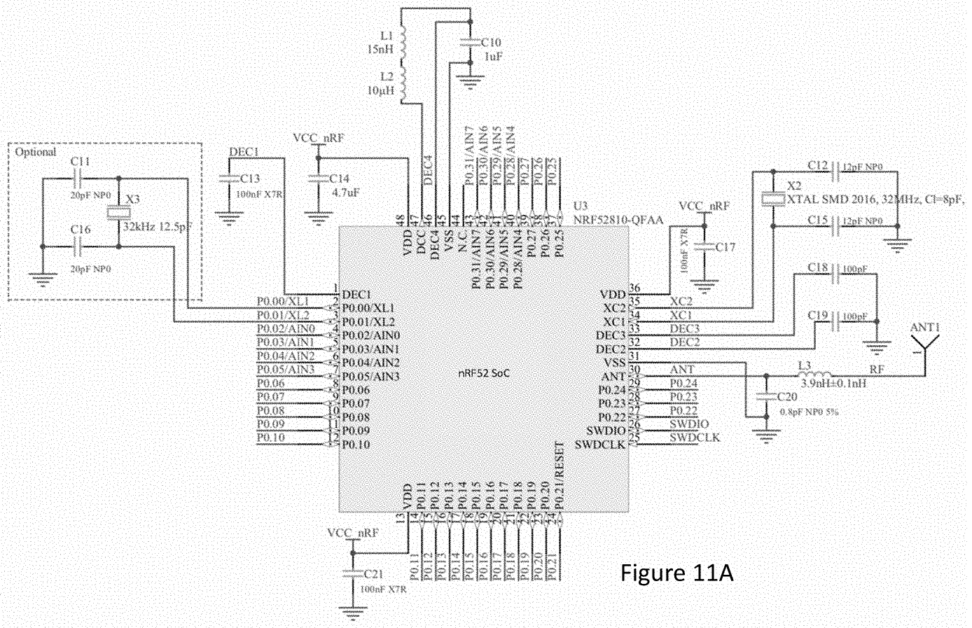

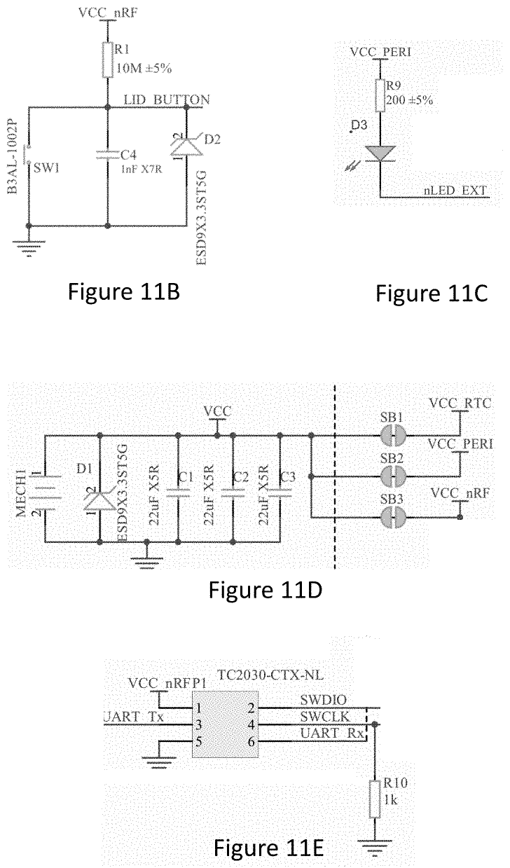

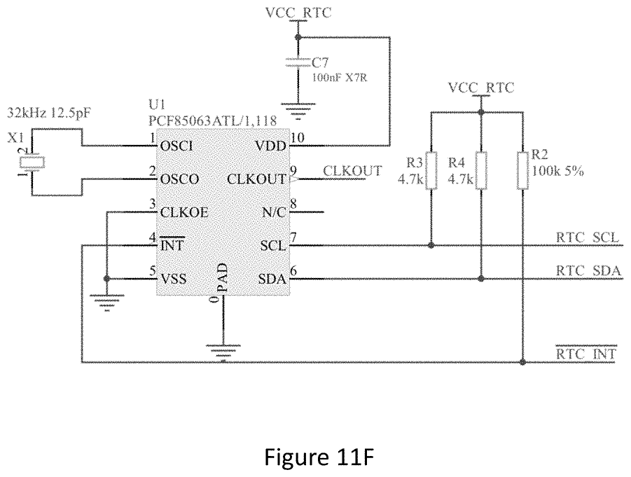

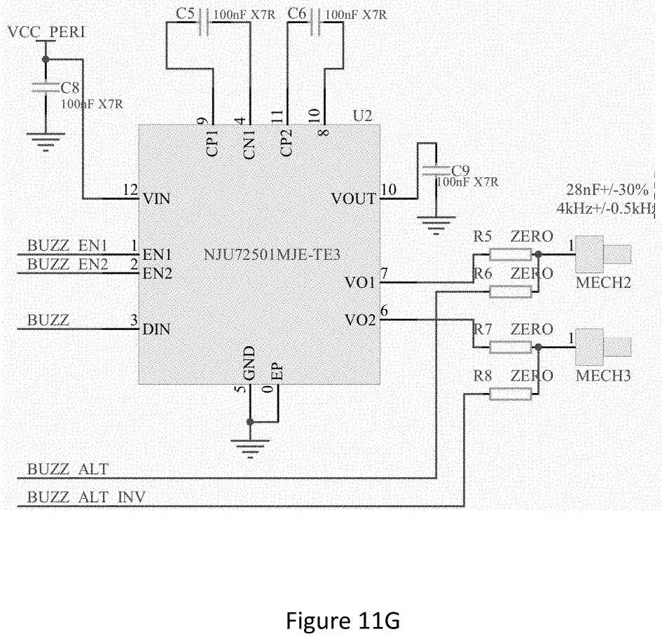

PCB 934 contains a plurality of electrical components optimized to fit within the cap unit 910, such as a microprocessor, a coin cell type battery 932, an antenna for wireless communications with the user device (FIG. 1, 110), and an internal contact sensor. In an exemplary embodiment, the internal contact sensor comprises a depressor switch mechanism detecting movement of the cap unit 910 between an opened and closed position, and for user double tapping of the cover 918 to order a prescription refill wirelessly and automatedly. A number of capacitors and transistors will also populate the PCB 934 (e.g. see FIGS. 11A-11G).

FIG. 11B illustrates an electric circuit schematic able to connect a button (or the internal contact sensor) to one of the general-purpose input-output pins of the system on a chip (SoC). The button-sensor can be used to detect the opening and closing of container unit 920 in response to the movement of the push-plate 914.

In order to achieve wireless connectivity with a user device (FIG. 1, 110), such as a smart phone or in-home base station, PCB 934 contains at least a wireless radio able to connect via the cellular or intranet network (FIG. 1, 105), and/or via a short range wireless radios (e.g. Bluetooth® versions 4.1-5 pairing). The antenna is a pre-packaged chip that is soldered onto the PCB 934, or it may be etched directly into the PCB. Cap unit 910 is thus capable of wireless communication via at least one of the following: Bluetooth radio; ANT radio, cellular radio, WiFi radio, Near Field Communication radio, or RFID. In one embodiment, the wireless radio is a 2.4 GHz low-energy radio, which transmits and receives a signal from user device 110 at distance of no more than about 1500 feet. In another embodiment, the wireless radio transmits either at a narrow band of 200 kHz spectrum, at 180 kHz, or between 1.4 and 20 MHz, at an uplink peak rate of not more than 10 Mbit/s. As used herein, the term “about” refers to 5 percent plus or minus the stated value.

FIG. 11A is an illustration of an exemplary electrical schematic of the PCB 934 comprising a nRF chip, such as a Nordic nRF51822-QFAC or the nRF52810-QFAA microprocessor chip. Both of these system on chips devices support 2.4 GHz wireless protocols (i.e. Bluetooth 4.1 or 5), have either a 16 MHz or 32 MHz microprocessor, and have onboard flash memory. Each chip comes pre-programmed with a unique MAC address stored in memory that is used when transmitting data wirelessly (e.g. to user electronic computing device 110, FIG. 1). This same MAC is used for unique device identification (e.g. a token). The antenna circuit is tuned for Bluetooth Low Energy and is capable of transmitting data approximately 100 meters.

FIG. 11E is an electrical circuit schematic of the programming port on the PCB 934, which is used to install firmware on the smart monitoring device 120 during manufacturing. It can also be used to read information from the device 120 during or after assembly.

FIG. 11F is an electrical circuit schematic of the PCB 934 real-time-clock (RTC). This optional circuit shows how a low power real-time-clock (RTC) can be connected to the SoC to keep accurate measurement of time. The RTC is also capable of triggering alarms on scheduled intervals.

### Battery

As illustrated in FIGS. 9D, 10A, and 10B, a battery connector 960 for holding battery 932, a microprocessor 110 having microprocessor memory and wireless antenna are each positioned to achieve a satisfactory connectivity range and battery life while maintaining a compact configuration. FIG. 11D exemplary electrical circuit schematic illustrating a power circuit that is used to connect the coin cell battery. PCB 934 may further comprise a battery holder clip 1103 shown in FIG. 10A on the top surface to fix battery 932 in position atop the PCB.

In one embodiment, the battery 932 is a non-rechargeable lithium battery cell (e.g. Coin cell battery (LiMnO2), CR2450 or larger capacity), measuring no more than 24 mm in diameter and no more than 5 mm in thickness. In another embodiment, the battery 932 measures between 20 and 25 mm in diameter, and between 3.0 and 5.2 mm in thickness. In a battery-optimized system, the wireless radio is likely to transmit between 25 and 1500 feet, and most likely between 50 and 400 feet. In another embodiment, battery 932 is a rechargeable battery. In another embodiment there may be no battery required, such as in the case where there is mechanism for receiving wireless power on the printed circuit board 934 itself.

### Microprocessor Memory and Token

As illustrated in FIGS. 10B and 11A the microprocessor 1010 comprises the antenna and the memory (inside the microprocessor unit 1010), which in an embodiment comprises a combination of flash memory and RAM memory, e.g. the data storage unit (FIG. 1, 123) and the data monitoring module (FIG. 1, 121) as non-transitory, tangible computer-readable storage medium on which databases and processor-executable software are embodied. Additionally, or alternatively, the smart monitor device 110 further comprises one or more removable memory cards or removable flash memory comprising the data storage unit (FIG. 1, 123). The data storage unit 123 further comprises token storage 125 for storing a token, e.g., the token comprising a Bluetooth® SIG-assigned UUID unique to the smart monitor device 120.

In an additional embodiment, the wireless radio transmitter of device 120 transmits, or sends wireless advertisement packets, at a first interval when there is no new data to sync, and at a second interval when there is un-synced data remaining in the memory. The electrical current supplied to the wireless radio transmitter from the power source (battery 932) and through the printed circuit board 934 is between about 0.5 mA and 25 mA when the wireless radio transmitter is advertising or is connecting to a user electronic computing device 110. And the electrical current supplied to the wireless radio transmitter from the power source (battery 932) and through the printed circuit board 934 is between about 0.5 mA and 10 mA when the wireless radio transmitter is receiving a wireless signal from the user electronic computing device 110.

Light Pipe: Referring again to FIG. 9A, the cover 918 may further comprise a transparent light pipe 922, which conducts light from a light-emitting source, such as a light emitting diode (LED) (see FIG. 10A, 1102) that is connected to the printed circuit board 934. LED 1102 is powered by the battery 932 on the printed circuit board (PCB, 934). A blinking light may indicate a container access alert or dose scheduled reminder, in addition to, or in lieu of, the piezo buzzer. FIG. 11C is an exemplary electrical circuit illustrating LED 1102, which can be used to notify or reminder the user.

Piezo Buzzer: Referring again to FIG. 9A, cover 918 further comprises a plurality of holes 921, to emit sound from the piezoelectric buzzer 931 located beneath the cover 918 within the internal cavity 923 of the cap unit 910. In an alternative embodiment, the piezoelectric buzzer 931 comprises a buzzer, speaker, or other sound-emitting component that is directly mounted on the PCB 934 or connected to it through another conductive mechanism. Preferably, the sound-emitting component emits a sound between 75 decibels and 95 decibels, such as a reminder to take a scheduled dose.

FIG. 11G illustrates an exemplary electrical schematic for the piezo buzzer on the printed circuit board, and comprising a 4 kHz buzzer that can be driven by the SoC either directly, or by using a charge pump, which increases the buzzer volume.

### Other Example Embodiments

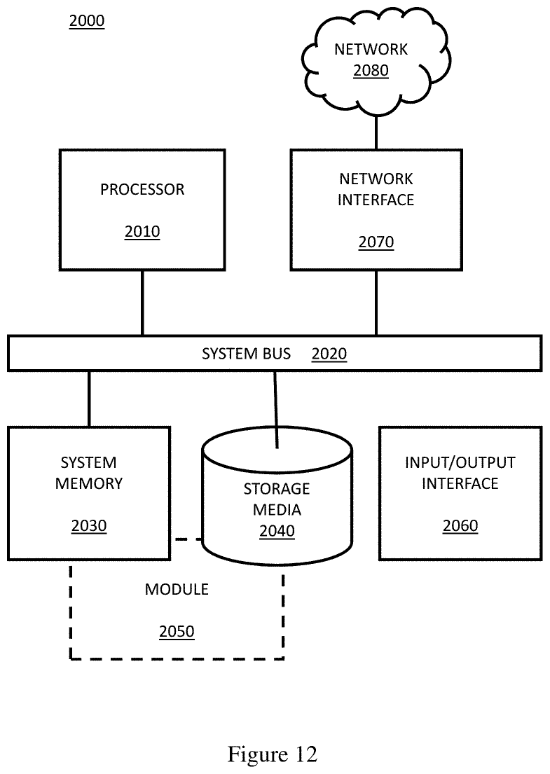

FIG. 12 depicts a computing machine 2000 and a module 2050 in accordance with certain example embodiments. The computing machine 2000 may correspond to any of the various computers, servers, mobile devices, embedded systems, or computing systems presented herein. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a vehicular information system, one more processors associated with a television, a customized machine, any other hardware platform, or any combination or multiplicity thereof. The computing machine 2000 may be a distributed system configured to function using multiple computing machines interconnected via a data network or bus system.

The processor 2010 may be configured to execute code or instructions to perform the operations and functionality described herein, manage request flow and address mappings, and to perform calculations and generate commands. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 2010 may be a single processing unit, multiple processing units, a single processing core, multiple processing cores, special purpose processing cores, co-processors, or any combination thereof. According to certain embodiments, the processor 2010 along with other components of the computing machine 2000 may be a virtualized computing machine executing within one or more other computing machines.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random access memory (“RAM”), static random access memory (“SRAM”), dynamic random access memory (“DRAM”), and synchronous dynamic random access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 may include, or operate in conjunction with, a non-volatile storage device such as the storage media 2040.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid sate drive (“SSD”), any magnetic storage device, any optical storage device, any electrical storage device, any semiconductor storage device, any physical-based storage device, any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth.

The module 2050 may comprise one or more hardware or software elements configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for operably coupling the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including mice, touch-screens, scanners, electronic digitizers, sensors, receivers, touchpads, trackballs, cameras, microphones, keyboards, any other pointing devices, or any combinations thereof. The I/O interface 2060 may couple the computing machine 2000 to various output devices including video displays, speakers, printers, projectors, tactile feedback devices, automation control, robotic components, actuators, motors, fans, solenoids, valves, pumps, transmitters, signal emitters, lights, and so forth.

The computing machine 2000 may operate in a networked environment using logical connections through the network interface 2070 to one or more other systems or computing machines across the network 2080. The network 2080 may include wide area networks (WAN), local area networks (LAN), intranets, the Internet, wireless access networks, wired networks, mobile networks, telephone networks, optical networks, or combinations thereof. The network 2080 may be packet switched, circuit switched, of any topology, and may use any communication protocol. Communication links within the network 2080 may involve various digital or an analog communication media such as fiber optic cables, free-space optics, waveguides, electrical conductors, wireless links, antennas, radio-frequency communications, and so forth.

The processor 2010 may be connected to the other elements of the computing machine 2000 or the various peripherals discussed herein through the system bus 2020. It should be appreciated that the system bus 2020 may be within the processor 2010, outside the processor 2010, or both. According to some embodiments, any of the processor 2010, the other elements of the computing machine 2000, or the various peripherals discussed herein may be integrated into a single device such as a system on chip (“SOC”), system on package (“SOP”), or ASIC device.

In situations in which the systems discussed here collect personal information about users, or may make use of personal information, the users may be provided with a opportunity or option to control whether programs or features collect user information (e.g., information about a user's social network, social actions or activities, profession, a user's preferences, or a user's current location), or to control whether and/or how to receive content from the content server that may be more relevant to the user. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over how information is collected about the user and used by a content server.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement an embodiment of the disclosed embodiments based on the appended flow charts and associated description in the application text. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

The example embodiments described herein can be used with computer hardware and software that perform the methods and processing functions described previously. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of various embodiments. Accordingly, such alternative embodiments are included in the inventions described herein.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

We claim:

1. A computer-implemented method for fulfilling a refill request for a user, comprising:
associating, by one or more computing devices, a product that is to be refilled with a smart monitor device;
determining, by one or more computing devices, one or more management parameters for the product, wherein the one or more management parameters are determined from one or more received product characteristics of the product and wherein the one or more one or more received product characteristics identify a number of refills available for the product;
associating, by the one or more computing devices, the smart monitor device with a user;
receiving, by the one or more computing devices and from the user device, a refill request, wherein the refill request identifies the user;
determining, by the one or more computing devices and based on the identity of the user and the one or more management parameters, a refill eligibility for the refill request; and,
initiating, by the one or more computing devices and based on a determination that the refill request is refill eligible, a refill for the refill request.

2. The computer-implemented method of claim 1, wherein associating the smart monitor device with the user comprises
obtaining, by the one or more computing devices, a first token associated with the smart monitor device,
associating, by the one or more computing devices, the first token with a user device of the user, and
authenticating, by the one or more computing devices and based on the first token, the user device of the user.

3. The computer-implemented method of claim 2, wherein associating the smart monitor device with the user further comprises recording, by the one or more computing devices, the first token in a token log.

4. The computer-implemented method of claim 3, wherein authenticating the user device further comprises:
receiving, by the one or more computing devices, a second token from the user device;
comparing, by the one or more computing devices, the received second token to the token log; and
identifying, by the one or more computing devices and based on the comparison of the received second token to the token log, a token match between the first token and the second token, wherein the token match authenticates the user device.

5. The computer-implemented method of claim 1, wherein determining the refill eligibility further comprises comparing, by the one or more computing devices, the refill request of the user with the one or more management paraments and wherein the comparison identifies an available refill for the product.

6. The computer-implemented method of claim 1, wherein the smart monitor device comprises a user control option and wherein the refill request is received in response a user input into the user control option.

7. The method of claim 1, further comprising notifying, by the one or more computing devices, the user that the refill request has been processed.

8. A system to provide a product refill, comprising:
a storage device;
a processor communicatively coupled to the storage device, wherein the processor executes application code instructions that are stored in the storage device to cause the system to:
associate a product that is to be refilled with a smart monitor device;
determine one or more management parameters for the product, wherein the one or more management parameters are determined from one or more received product characteristics of the product and wherein the one or more one or more received product characteristics identify a number of refills available;
associate the smart monitor device with a user;
receive, from the user device, a refill request, wherein the refill request identifies the user;
determine, based on the identity of the user and the one or more management parameters, a refill eligibility for the refill request; and,
initiate, based on a determination that the refill request is refill eligible, a refill for the refill request.

9. The system of claim 8, wherein associating the smart monitor device with the user comprises obtaining a first token associated with the smart monitor device, associating, the first token with a user device of the user, and authenticating the user device of the user based on the first token.

10. The system of claim 9, wherein associating the smart monitor device with the user further comprises recording the first token in a token log.

11. The system of claim 10, wherein authenticating the user device further comprises:
receiving a second token from the user device;
comparing the received second token to the token log; and
identifying, based on the comparison of the received second token to the token log, a token match between the first token and the second token, wherein the token match authenticates the user device.

12. The system of claim 8, wherein determining the refill eligibility further comprises comparing the refill request of the user with the one or more management paraments and wherein the comparison identifies an available refill for the product.

13. The system of claim 8, wherein the smart monitor device comprises a user control option and wherein the refill request is received in response a user input into the user control option.

14. The system of claim 8, further comprising notifying the user that the refill request has been processed.

15. A computer program product, comprising:
a non-transitory computer-readable storage device having computer-readable program instructions embodied thereon that when executed by a computer cause the computer to provide a product refill, the computer-executable program instructions comprising:
computer program instructions to associate a product that is to be refilled with a smart monitor device;
computer program instructions to determine one or more management parameters for the product, wherein the one or more management parameters are determined from one or more received product characteristics of the product and wherein the one or more one or more received product characteristics identify a number of refills available;
computer program instructions to associate the smart monitor device with a user;
computer program instructions to receive, from the user device, a refill request, wherein the refill request identifies the user;
computer program instructions to determine, based on the identity of the user and the one or more management parameters, a refill eligibility for the refill request; and,
computer program instructions to initiate, based on a determination that the refill request is refill eligible, a refill for the refill request.

16. The computer program product of claim 15, wherein associating the smart monitor device with the user comprises obtaining a first token associated with the smart monitor device, associating, the first token with a user device of the user, and authenticating the user device of the user based on the first token.

17. The computer program product of claim 16, wherein associating the smart monitor device with the user further comprises recording the first token in a token log.

18. The computer program product of claim 17, wherein authenticating the user device further comprises:
receiving a second token from the user device;
comparing the received second token to the token log; and
identifying, based on the comparison of the received second token to the token log, a token match between the first token and the second token, wherein the token match authenticates the user device.

19. The computer program product of claim 15, wherein determining the refill eligibility further comprises comparing the refill request of the user with the one or more management paraments and wherein the comparison identifies an available refill for the product.

20. The computer program product of claim 15, wherein the smart monitor device comprises a user control option and wherein the refill request is received in response a user input into the user control option.
